(19) United States
(12) Patent Application Publication
(10) Pub. No.: US 2025/0014707 A1
(43) Pub. Date: Jan. 9, 2025

(54) METHOD AND SYSTEM FOR AUTOMATIC MONITORING OF DIABETES RELATED TREATMENTS BASED ON INSULIN INJECTIONS

(71) Applicant: DREAMED DIABETES LTD., Petah Tivka (IL)

(72) Inventors: Yaron MATIAS, Giv'atayim (IL); Anat COHEN, Tel Aviv (IL); Tomer SEGALL, Tel Aviv (IL); Ilana KLO-VATCH, Petah Tikva (IL); Ido MULLER, Ness-Ziona (IL)

(21) Appl. No.: 18/708,945

(22) PCT Filed: Nov. 9, 2021
(86) PCT No.: PCT/IL2021/051324

§ 371 Date: May 9, 2024

(51) Int. Cl. G16H 20/17; A61M 5/172

(52) U.S. Cl. CPC G16H 20/17; A61M 5/1723; A61M 2202/0486

(57) ABSTRACT

Some embodiments relate to a method and system for automatic monitoring of diabetes related treatments based on insulin injections. The technique enables to receive at least raw log glucose data and provide a recommended patient-specific insulin injections treatment plan (i.e. automatic individualized recommendations to change insulin therapy) to patients on insulin, either on long acting insulin only or on Multiple Daily Injections (i.e. MDI treatment), based on the data analysis.

### CROSS-REFERENCE TO RELATED APPLICATIONS

This application is a national phase filing under 35 C.F.R. § 371 of and claims priority to PCT Patent Application No. PCT/IL2021/051324, filed on Nov. 9, 2021.

### TECHNOLOGICAL FIELD

This presently disclosed subject matter is in the field of monitoring diabetes-related treatment and relates to a method and system for automatic monitoring of diabetes related treatments based on insulin injections.

### BACKGROUND ART

References considered to be relevant as background to the presently disclosed subject matter are listed below:
US patent publication No. 2018/200434
US patent publication No. 2013/165901

Acknowledgement of the above references herein is not to be inferred as meaning that these are in any way relevant to the patentability of the presently disclosed subject matter.

### BACKGROUND

The introduction of new technologies such as software for big data collection, continuous glucose monitoring, smart pumps, insulin connected pen and glucometers and medical Apps and Software, aims to provide patients with more tools to self-manage their disease, and healthcare professionals with the ability to better support and treat patients. Nevertheless, it requires the healthcare professionals to apply a new spectrum of theoretical knowledge and practical skills. More specifically, there is a need in the art in assisting patients with an automatic monitoring technique fulfilling the day-to-day demands of diabetes management based on insulin injections.

### General Description

Typically, a patient receives an insulin injections treatment plan from a physician for a certain time period of several months. He should then consult the physician periodically to adjust the plan according to his personal recordings and subjective feelings on the fitting of this plan to his personal daily routine. This process has a lot of drawbacks since it totally depends on the patient's manual recording, and adjustment of the plan cannot be personalized and accurate. Some techniques provide a real-time adjustment of a treatment plan in which the changes in glucose level are continuously recorded and some instantaneous corrections of the currently measured glucose level are proposed to the patient. However, these approaches do not change the treatment plan itself and therefore are not adapted to the specific patient daily routine.

The technique of the presently disclosed subject matter enables to receive at least raw log glucose data and provide a recommended patient-specific insulin injections treatment plan (i.e. automatic individualized recommendations to change insulin therapy) to patients on insulin, either on long acting insulin only or on Multiple Daily Injections (i.e. MDI treatment), based on the data analysis.

According to a broad aspect of the presently disclosed subject matter, there is provided a monitoring system for use with diabetic treatment management based on insulin injections. In this connection, it should be noted that the term “insulin injections” hereinafter refer to ‘Basal only’ treatment or MDI treatment. “Basal only” treatment or long acting insulin only treatment is a type of treatment in which the patients are using only basal injections (long acting insulin) and are not using any other type of insulin. Therefore, these patients (usually of Type 2 Diabetes) only have a basal plan. MDI treatment is a type of treatment in which the patient uses both basal (i.e. long acting insulin) and bolus (i.e. short acting insulin), and therefore their treatment is composed of two kinds of plans: one for the basal and a second for the boluses (as described in details further below). Therefore, the control unit is configured and operable to automatically determining a recommended patient-specific insulin injections treatment plan based on basal only and/or a recommended patient-specific Multiple Daily Injections (MDI) treatment plan. The monitoring system comprises a communication interface configured and operable to permit access to raw log glucose data being indicative of blood glucose levels over a certain time period; wherein the certain time period being at least one daily period and at least one of an existing patient-specific insulin injections treatment plan or user data being indicative of at least one of event or insulin delivery over the certain time period; and a control unit configured and operable for receiving and processing the raw glucose log data with at least one of the existing insulin injections treatment plan or the user data and automatically determining a recommended patient-specific insulin injection treatment plan wherein the recommended patient-specific treatment plan comprises individualized insulin dosing injection parameters data over the certain time period, wherein the individualized insulin dosing injection parameters data comprises a basal plan and/or a bolus plan and/or suggestions for personalized diabetes management tips. The patient-specific insulin injections treatment plan comprises individualized insulin dosing injection parameters data over a period time of at least one day (i.e. 24 hours). In this connection it should be understood that the insulin injections treatment plan provided by the presently disclosed subject matter defines a routine diabetes plan not limited to a specific time period. The technique of the presently disclosed subject matter analyses at least the raw log glucose data and adjusts the entire patient-specific insulin injections treatment plan to be optimized to the patient daily routine for a non-limited time period. To be able to provide automatic individualized recommendations to change insulin therapy, the raw log data should comprise data collected during a time period of at least one day (e.g. a plurality of days) prior to the recommendation time. The technique of the presently disclosed subject matter analyzes retrospective data and provides recommendation on changes for the treatment plan (this is not a real-time system). The raw log glucose data is indicative of at least one blood glucose pattern defining blood glucose levels over a certain time period and may comprise direct glucose measurement (i.e. measured data collected by a continuous glucose monitoring (CGM) system and/or a glucometer and/or Flash Glucose Monitoring (FGM)) and/or also optionally a user's input (i.e. glucose data manually collected over the certain time period using for example an application). The system receives and analyzes individual's diabetes data including glucose levels, in combination with at least one of (i) an existing patient-specific insulin injections treatment plan or (ii) user data being indicative of at least one of (1) an event such as meals consumed during the certain time period, physical activity, illness etc. or of (2) the insulin delivery history (i.e. insulin delivery over the certain time period).

The recommended insulin injections treatment plan may comprise the following parameters depending on the insulin injections treatment plan received by the system of the presently disclosed subject matter: a basal plan (i.e. daily long acting insulin injection plan) and/or a bolus plan (including dosage and timing of short acting insulin parameters such as (1) Carbohydrate Ratio (CR), Correction Factor (CF) and glucose target; and/or (2) Fixed dose, CF and glucose target; and/or (3) Meal Size Estimation, CF and glucose target and/or (4) Sliding scale (SSI)) and/or (5) suggestions for personalized diabetes management tips (such as timing of meal boluses, bolus delivery compliance or treatment alert to the patient or to health care professionals (HCP)). In this connection, it should be noted that the term “bolus” refers to the dosage of insulin intended to “cover” a meal and/or to make a large glucose level correction. The term “Carbohydrate Ratio (CR)” may refer to one insulin unit per gr of carbohydrates or per exchange of grams. The term “Correction Factor (CF)” may refer to the expected amount of glucose level decrease in the blood, given 1 unit of insulin. The term “SSI” may refer to a type of bolus plan in which the amount of insulin that should be delivered is according to predetermined glucose ranges. The term “Meal Size Estimation” may refer to fix amount of insulin per size of meal, where the size of the meal may be defined i.e. as ‘small’/‘medium’/‘large’.

Therefore, in some embodiments, the control unit is configured and operable for automatically determining the basal plan including an amount of long acting insulin dosage, and/or bolus plan including an amount of short acting insulin dosage. Additionally or alternatively, the control unit may be configured and operable for automatically determining an amount of long acting insulin dose comprising analyzing the blood glucose pattern to define a certain time of injecting the long acting insulin dose and a certain number of required doses of the basal insulin. Additionally or alternatively, the control unit may be configured and operable for automatically determining the bolus plan including (1) carbohydrate ratio (CR), correction factor (CF) and glucose target; and/or (2) fixed dose, CF and glucose target; and/or (3) meal size estimation, CF and glucose target and/or (4) Sliding Scale (SSI) according to the time of the day, over the certain period of time. Additionally or alternatively, the control unit may be configured and operable for automatically determining and providing the personalized diabetes management tips to at least one of the patient or a physician, wherein the personalized diabetes management tips comprise textual output data being indicative of at least one of timing of meal boluses, bolus delivery compliance, or a treatment alert.

Comparing the above to the conventional approach, it should be understood that the presently disclosed subject matter eliminates a need for a physician to conduct any retrospective analysis (i.e., look at the data during the clinical visit) and subjectively conclude how to change the insulin injections treatment plan based on this information. This is advantageous because practically not all physicians have the needed expertise to fulfill this task properly. In addition, for those who have the needed expertise, this task is very time consuming. Sometimes analyzing the data becomes very difficult due to the fact the data has no clear pattern visible/identifiable to the human eye in order to derive conclusions and propose the appropriate insulin injections treatment plan.

Therefore, the presently disclosed subject matter addresses the challenge of replacing the trained physician's retrospective analysis of the patient's input by providing a monitoring system which is capable of properly analyzing the raw log data input and optionally user data. Such a monitoring system of the presently disclosed subject matter may organize the data (i.e. isolate the informative essence from the subordinate) and provide recommendations to the insulin injections treatment plan in order to improve glucose control in the period of time that follows the recommendation. The input to the system may include solely the stored raw log data obtained over a certain time window being indicative of glucose levels and user data being indicative of insulin delivery and/or other events such as meal, physical activity, etc.

According to another broad aspect of the presently disclosed subject matter, there is provided a monitoring system comprising a communication interface configured and operable to permit access to raw log glucose data being indicative of blood glucose levels over a certain time period; wherein the certain time period being at least one daily period; and a control unit configured and operable for receiving and processing the raw glucose log data to identify the existing patient-specific treatment plan according to the user's input. This monitoring system may be used together with the monitoring system defined above, or as a separate entity. In this case, the monitoring system is able to automatically identify the current basal plan based on the basal records, and the bolus plan in case of SSI, based on the bolus records.

According to another broad aspect of the presently disclosed subject matter, there is provided a monitoring system comprising a communication interface configured and operable to permit access to raw log glucose data being indicative of blood glucose levels over a certain time period; wherein the certain time period being at least one daily period; and a control unit configured and operable for receiving and processing the raw glucose log data to identify a start point and an end point of a certain inactive time period being indicative of an absence of insulin delivery and meal consumption. This monitoring system may be used together with the monitoring system(s) defined above or as a separate entity. In this case, the monitoring system is able to automatically identify at least one inactive period such as a night pattern. More specifically, the system may be able to identify the beginning and the end of the night period by analyzing the glucose pattern being measured, the injection or physical activity (PA) records provided by the user's input. The technique is able to provide specific recommendations to a patient-specific basal plan, based on the nighttime window.

According to another broad aspect of the presently disclosed subject matter, there is provided a monitoring system for automatically identifying patient-specific changes to the insulin injections treatment plan (bolus and/or basal) based on glucose levels only. The monitoring system comprises a communication interface configured and operable to permit access to raw log glucose data being indicative of blood glucose levels over a certain time period; wherein the certain time period being at least one daily period; and an existing patient-specific insulin injections treatment plan. The control unit configured and operable for processing the raw log glucose data to thereby identify, in the raw log glucose data, event data being indicative of at least one of insulin delivery or at least one meal consumed in a certain time period. This monitoring system may be used together with the monitoring system(s) defined above, or as a separate entity.

According to another broad aspect of the presently disclosed subject matter, there is provided a monitoring system being able to perform behavioral pattern analysis. This monitoring system may be used together with the monitoring system(s) defined above or as a separate entity. This technique enables to classify/categorize events relating or not to the existing patient-specific insulin injections treatment plan and filtering out the events not relating to the existing patient-specific insulin injections treatment plan. For example, boluses that the system found based on the glucose levels may be filtered out in case there was a hypoglycemia event before the estimated bolus time. Thus, such a bolus event may not affect the estimation of the insulin injections treatment plan. The different events may also have different weights in the decision treatment plan proposed to the patient. The control unit may thus be configured and operable to automatically determine a patient-specific bolus treatment plan based on event data by giving different weights to the classified events. The different events may change the dosage of the patient-specific treatment plan or provide suggestions for personalized diabetes management tips (such as timing of meal boluses, bolus delivery compliance or over-treating events of hypoglycemia).

In some embodiments, the communication interface comprises a user interface to permit user input of the user data comprising data being indicative of insulin delivery over the certain time, meals consumed, at least a part of the raw log glucose data, exercise intensity or any other textual data being indicative of the user's condition. The communication interface may be configured to permit input of at least a part of the raw log glucose data directly from an external device. The external device may comprise at least one of the following: a measurement device, a storage device, and a drug injection device. The measurement device may comprise at least one of a continuous glucose monitor and a glucometer.

According to another broad aspect of the presently disclosed subject matter, there is provided a monitoring system being able to perform a data integration between the raw log glucose data corresponding to a glucose pattern averaged for a certain period of time of at least a plurality of days, and the user data including event data being based on user's input. This monitoring system may be used together with the monitoring system(s) defined above or as a separate entity. In some embodiments, the monitoring system is able to synchronize between the different raw log glucose data (CGM, FGM, SMBG and manual glucose entries using an application) and optionally also between the raw log glucose data and the user data. The control unit may thus be configured and operable to synchronize between the measured data generated by a plurality of external devices and/or to receive and process the raw log glucose data and the user data and to synchronize between the raw log glucose data and the user data. For example, the monitoring system is able to synchronize between a plurality of external devices such as a CGM and glucometer. This enables to improve the timing precision and the glucose values of the glucose pattern, as well as the data integration as described above.

According to another broad aspect of the presently disclosed subject matter, there is provided a computer program recordable on a storage medium and comprising a machine-readable format, the computer program being configured and operable to, when being accessed, carry out the method as will be defined below. The computer program product may comprise a non-transitory tangible computer readable medium having computer readable program code embodied therein, the computer readable program code adapted to be executed to implement a method as described below. The computer program is configured and operable, when being accessed, to carry out the following: receiving raw log glucose data being indicative of at least one blood glucose pattern defining blood glucose levels over a certain time period; wherein the certain time period being at least one daily period; and at least one of an existing patient-specific insulin injections treatment plan or a user data comprising at least one of data being indicative of insulin delivery over the certain time period or of data being indicative of meals consumed during the certain time period; processing the raw log glucose data with at least one of the existing insulin injections treatment plan or the user data to automatically determine a recommended patient-specific insulin injections treatment plan wherein the recommended patient-specific insulin injections treatment plan comprises individualized insulin dosing injection parameters data over the certain time period, wherein the individualized insulin dosing injection parameters data comprises a basal plan or a bolus plan or suggestions for personalized diabetes management tips. In some embodiments, the control unit is configured and operable to correlate between the raw log glucose data, insulin records and the existing patient-specific insulin injections treatment plan to validate the existing patient-specific insulin injections treatment plan. The system can thus validate that the current insulin injections treatment plan is suitable for the patient and no further changes are required (meaning that glucose levels are good).

In some embodiments, the control unit is configured and operable to receive and process the measured data to identify the user data being indicative of insulin delivery over the certain time period or meals consumed during the certain time period.

According to another broad aspect of the presently disclosed subject matter, there is provided a method of automatic monitoring of diabetes-related treatment of a patient based on insulin injections. The method comprises receiving raw log glucose data being indicative of at least one blood glucose pattern defining blood glucose levels over a certain time period; wherein the certain time period being at least one daily period; and at least one of an existing patient-specific insulin injections treatment plan or a user data comprising at least one of data being indicative of insulin delivery over the certain time period or of data being indicative of meals consumed during the certain time period; processing the raw log glucose data with at least one of the existing insulin injections treatment plan or the user data to automatically determine a recommended patient-specific insulin injections treatment plan; wherein the recommended patient-specific insulin injections treatment plan comprises individualized insulin dosing injection parameters data over the certain time period, wherein the individualized insulin dosing injection parameters data comprises a basal plan or a bolus plan or suggestions for personalized diabetes management tips.

In some embodiments, automatically determining a patient-specific insulin injections treatment plan comprises automatically determining a basal plan including at least one of timing of insulin delivery, an amount of long acting insulin dosage, number of required doses of basal insulin, and time for basal insulin injection.

In some embodiments, automatically determining an amount of long acting insulin dose comprises analyzing the glucose pattern to define a certain time of injecting the long acting insulin dose and a certain number of required doses of the basal insulin.

In some embodiments, automatically determining the bolus plan includes determining (1) CR, CF and glucose target; and/or (2) fixed dose, CF and glucose target, and/or (3) meal size estimation, CF and glucose target and/or (4) SSI according to the time of day over the certain period of time.

In some embodiments, automatically determining the personalized diabetes management tips comprises providing the personalized diabetes management tips to at least one of the patient or a physician, wherein the personalized diabetes management tips comprise textual output data comprising at least one of timing of meal boluses, bolus delivery compliance, overtreating hypoglycemia, bolus stacking, or other treatment alert. In some embodiments, receiving user data comprises receiving user input of the user data comprising data being indicative of at least of the insulin delivery over the certain time, meals consumed, at least a part of the raw log glucose data, exercise intensity or any other textual data being indicative of the user's condition.

In some embodiments, receiving measured data comprises receiving input of at least a part of the raw log glucose data from an external device. Receiving measured data may also comprise receiving measured data from a plurality of external devices.

In some embodiments, processing the raw log glucose data comprises synchronizing between the measured data generated by the plurality of external devices.

In some embodiments, processing the raw log glucose data and the user data comprises synchronizing between the raw log glucose data and the user data.

In some embodiments, the method further comprises correlating between the raw log glucose data, insulin records and the existing patient-specific insulin injections treatment plan to validate the existing patient-specific insulin injections treatment plan.

In some embodiments, processing the raw log glucose data comprises receiving and processing the measured data to identify the user data being indicative of insulin delivery over the certain time period or meals consumed during the certain time period.

In some embodiments, processing the raw log glucose data comprises identifying a start point and an end point of a certain inactive time period being indicative of an absence of insulin delivery and meal consumption.

In some embodiments, processing the raw log glucose data comprises identifying in the raw log glucose data event data being indicative of at least one of insulin delivery or at least one meal consumed in a certain time period; classifying the event data in different events either relating or not relating to the existing patient-specific insulin injections treatment plan and filtering out the events not relating to the existing patient-specific insulin injections treatment plan. Automatically determining of a patient-specific insulin injections treatment plan may comprise processing the event data by giving different weights to the classified event data.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic block diagram illustrating, in a non-limiting manner, the system for monitoring a diabetic treatment of a diabetic patient based on insulin injections;

FIG. 2 is a schematic block diagram illustrating, in a non-limiting manner, the possible input and output of the monitoring system according to some embodiments of the presently disclosed subject matter;

FIG. 3 is a flow chart illustrating, in a non-limiting manner, a possible method for monitoring a diabetic treatment of a diabetic patient based on insulin injections according to some embodiments of the presently disclosed subject matter;

FIG. 4 graphically illustrates an example of a processed glucose pattern allowing providing a recommendation for changing the insulin treatment plan according to some embodiments of the presently disclosed subject matter;

FIG. 5 graphically illustrates an example of a bolus amount versus the glucose level at the time of the bolus;

FIGS. 6A-6C graphically illustrate three different examples in which an automatic identification and classification of a bolus event is based on glucose levels only according to some embodiments of the presently disclosed subject matter;

FIG. 7 graphically illustrates an example of a raw data (glucose and insulin) along with tables describing the recommendation for several days;

FIGS. 8A-8B graphically illustrate an example of a glucose pattern allowing providing a recommendation for changing the insulin treatment plan based on glucose data only (CGM) according to some embodiments of the presently disclosed subject matter;

FIG. 9A-9B graphically illustrate an example of a glucose pattern allowing providing a recommendation for changing the insulin treatment plan based on glucose data only (SMBG) according to some embodiments of the presently disclosed subject matter;

FIGS. 10A-10B graphically illustrate an example in which two sources of glucose data are synchronized according to some embodiments of the presently disclosed subject matter;

FIGS. 11A-11B graphically illustrate an example in which both bolus records and glucose levels are used for determining the fasting period according to some embodiments of the presently disclosed subject matter;

FIG. 12 graphically illustrates an example in which only the glucose levels are used to determine the fasting period according to some embodiments of the presently disclosed subject matter;

FIG. 13 graphically illustrates a possible way the system may use the pre-fasting and fasting glucose levels to decide on the basal percentages of change (POC) and weights for each day separately according to some embodiments of the presently disclosed subject matter;

FIG. 14 graphically illustrates an example in which only the fasting glucose levels are used to determine the long acting insulin injection plan according to some embodiments of the presently disclosed subject matter;

FIG. 15 graphically illustrates a possible way the system may collect bolus events in one period of the day, decide on their POC and weights, and estimate the POC for the bolus plan for that specific period of the day according to some embodiments of the presently disclosed subject matter; and

FIGS. 16A-16H graphically illustrates an example in which the system detects a behavioral pattern of overtreating hypoglycemia according to some embodiments of the presently disclosed subject matter.

### DETAILED DESCRIPTION OF EMBODIMENTS

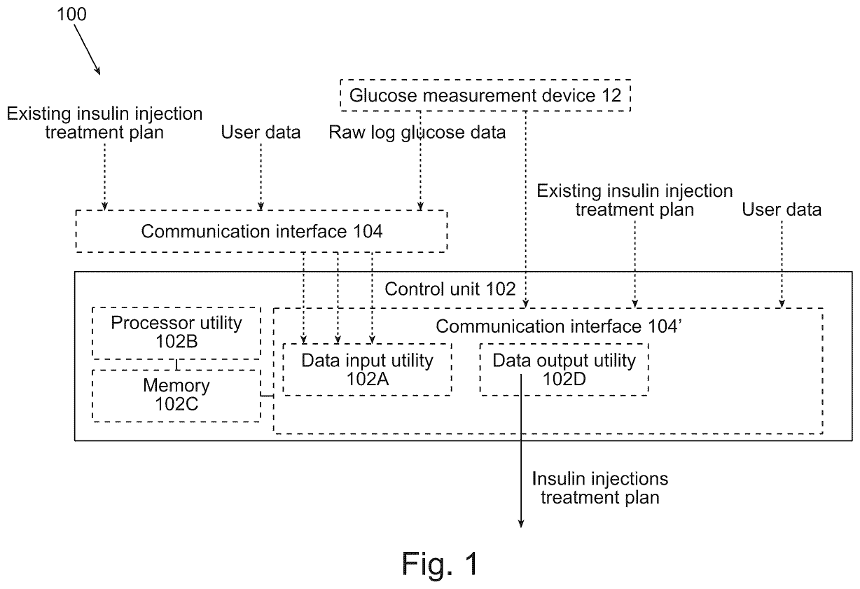

Reference is made to FIG. 1 exemplifying a simplified schematic block diagram illustrating a non-limiting example of a monitoring system 100 for carrying out monitoring of a diabetic treatment of a diabetic patient based on insulin injections in accordance with one embodiment of the presently disclosed subject matter. The monitoring system 100 is implemented by a software product configured for assisting in diabetic treatment of a diabetic patient based on insulin injections. to patients on insulin, either on long acting insulin only or on Multiple Daily Injections (MDI) therapy. This system is intended as a tool for physicians and/or for patients and/or caregivers. The monitoring system 100 comprises a control unit 102 being typically processor-based and including inter alia a memory utility 102C, data input and output utilities (102A and 102D), and a data processor utility 102B. The monitoring system 100 may be associated with a glucose measurement device 12 (e.g. Continuous Glucose Monitoring (CGM) or Flash Glucose Monitoring (FGM) or Self Monitoring Blood Glucose (SMBG, or glucometer)). Raw log glucose data, being indicative of blood glucose (BG) levels over a certain time period from measurement device 12, whether directly measured or provided by the user, enters, through a communication interface 104, a data processing and analyzing utility 102B of a control unit 102 of the monitoring system 100 and optionally also, typically, a memory utility 102C for storage. The user of the monitoring system of the presently disclosed subject matter may be the patient or health care professionals (HCP). More specifically, the raw log glucose data may include data indicative of the time and the value. The raw log data can be obtained or received from stored data (from a memory utility which may be associated with a remote computer system/database or with the measurement device of the patient). The presently disclosed subject matter utilizes data records, being raw log data, obtainable from a storage apparatus used to record the data and possibly other information during the everyday routine of the treated patient i.e. recordation of everyday routine. Data collected at the patient's everyday routine activity is different from that gathered while intentionally guiding the patient's activity. For the purposes of the presently disclosed subject matter, the raw log data may be continuously accumulated without any special attention of the monitored patient (other than being connected to the monitoring unit), as well as without any special attention of clinical personnel. Recording these measurements over time is performed as a part of a monitoring phase, in any known suitable technique, which by itself does not form part of the presently disclosed subject matter. It is important to note that this raw log data is gathered over a time interval of at least one day (e.g. several days) during everyday activity of the patient. Raw log data therefore includes a digital representation of measured signal(s) from the analyte sensor directly related to the measured glucose and data that was recorded by the patient as insulin delivery, meal(s) consumed, physical activity, glucose levels and insulin plans. For example, the raw data stream is digital data converted from an analog signal representative of the glucose concentration at a point in time, or a digital data representative of meal consumption at a point in time. The terms broadly encompass a plurality of time spaced data points from a substantially continuous analyte sensor (or continuous glucose sensor), each of which comprises individual measurements taken at time intervals ranging from fractions of a second up to, for example, 2, 4, or 10 minutes or longer. The time-spaced data points, in some embodiments, adhere to a preprogrammed sampling pattern. For example, monitoring system 100 is configured and operable to analyze the patient's glucose control performance based on glucose readings and pre-defined time buckets according to the time of the day. The monitoring system 100 may produce, in an aggregated format, the time of day (in HH:MM-HH:MM) of the following glucose patterns: (1) high pattern-based on glucose percentile profile, i.e. 25th, 50th, 75th and 90th percentiles; and/or (2) low pattern-based on hypo events and the percentile profile (10^{th} and or 25^{th} percentiles-should they exist).

For example, the monitoring system 100 is capable to receive data from a Data Management System (DMS) including at least one of: data from the glucose measurement devices; the insulin injections plan; the time stamp and the amount of delivered insulin; the carbs consumed; the reported glucose levels (i.e. the glucose levels that were manually uploaded to the system by the patient using an application or by other means); the glucose units; the injection type i.e. basal or bolus; the insulin type i.e. rapid or regular. The DMS may be a DMS, being a platform to which the patient can upload the data, and the physician can view the data. The insulin injections plan includes (i) the basal plan including time of day, dosage [U] and insulin type and/or (ii) the bolus plan (based on the treatment type) including either:
Carbohydrate Ratio (CR), Correction Factor (CF) and glucose target
Fixed dose, CF and glucose target
Meal Size Estimation, CF and glucose target
Sliding Scale (SSI)
   based on the treatment type for all day parts (e.g. morning, afternoon, evening, bedtime).

The control unit 102 provides a retrospective analysis of raw log data, which is input into the control unit 102 while in a machine-readable format, via a communication interface 104. Communication interface 104 is appropriately configured for connecting the processor utility 102B, via wires or wireless signal transmission (e.g. via communication network(s)), to either a measurement device supplying the raw log data or to an external memory (database) where such raw log data have been previously stored (being supplied to from measurement device(s)). Communication interface 104 may be a separate utility from control unit 102 or may be integrated within control unit 102 as illustrated by communication interface 104′. When communication interface 104 is a separate unit from control unit 102, control unit 102 may comprise a transceiver permitting to be connected to communication interface 104 and to transmit and/or receive data. When communication interface 104′ is integrated within control unit 102, it may include the data input utility 102A and data output utility 102D of control unit 102. The control unit 102 may comprise a transceiver permitting to be connected to a communication unit and to transmit and/or receive data. Communication interface 104 also permits access to an existing patient-specific insulin injections treatment plan or a user data being indicative of at least one of an event or insulin delivery over the certain time period. The raw log data may thus include one of glucose measurement device reading/levels as a function of time, and the user data may include event data as a function of time, and insulin delivery data as a function of time. User data may also include activity diary, such as meals, physical activity, sleep time etc. More specifically, user data, such as but not limited to: time of day, meals description e.g. amount of carbohydrates (CHO), percentage of protein (P), percentage of fat (F), human selection of a specific meal description from a list (i.e. breakfast, lunch or snack), physical activity (PA) whether accomplished or planned, sleep time, and presence of illness may be entered into the memory utility 102C by the user using communication interface 104 or another suitable user interface (not shown).

Therefore, monitoring may take into account user data i.e. inputs entered to the control unit 102 by the user such as but not limited to some or all of: the amount of carbohydrates consumed, other ingredients of the meal (e.g. percentage of fat and/or protein), specific event patient is participating in, function of a specific time of day (e.g. sleep, meal, exercise, disease). To determine the compounds of each meal, the control unit 102 may present the user with pre-programmed meals including details of ingredients. The event may be at least one of sleep, meal, exercise, disease event, or rest.

The control unit 102 may be configured as an electronic module for collecting and processing data. It should be noted that all required operations may be controlled by means of a processing utility, such as a DSP, microcontroller, FPGA, ASIC, etc., or any other conventional and/or dedicated computing unit/system. The term “processing utility” should be expansively construed to cover any kind of electronic device with data processing capabilities, including, by way of non-limiting example, personal computers, servers, computing systems, communication devices, processors (e.g. digital signal processor (DSP), microcontrollers, field programmable gate array (FPGA), application specific integrated circuit (ASIC), etc.) and other electronic computing devices. The processing utility may comprise a general-purpose computer processor, which is programmed in software to carry out the functions described hereinbelow. Also, operations in accordance with the teachings herein may be performed by a computer specially constructed for the desired purposes or by a general purpose computer specially configured for the desired purpose by a computer program stored in a computer readable storage medium. The different elements of the control unit (electronic unit and/or mechanical unit) are connected to each other by wires or are wireless. The software may be downloaded to the processing utility in electronic form, over a network, for example, or it may alternatively be provided on tangible media, such as optical, magnetic, or electronic memory media. Alternatively or additionally, some or all of the functions of the control unit may be implemented in dedicated hardware, such as a custom or semi-custom integrated circuit, or a programmable digital signal processor (DSP). The terms control unit and processor utility are used herein interchangeably, and furthermore refer to a computer system, state machine, processor, or the like, designed to perform arithmetic or logic operations using logic circuitry that responds to and processes the instructions that drive a computer.

The techniques and system of the presently disclosed subject matter can find applicability in a variety of computing or processing environments, such as computer or process-based environments. The techniques may be implemented in a combination of software and hardware. The techniques may be implemented in programs executing on programmable machines such as stationary computers being configured to obtain raw log data, as has also been described above. Program code is applied to the data entered using the input device to perform the techniques described and to generate the output information. The output information can then be applied to one or more output devices.

Each program may be implemented in a high-level procedural or object-oriented programming language to communicate with a processed based system. However, the programs can be implemented in assembly or machine language, if desired.

In other embodiments, the methods and systems of the presently disclosed subject matter can be utilized over a network computing system and/or environment. A number of computer systems may be coupled together via a network, such as a local area network (LAN), a wide area network (WAN) or the Internet. Each method or technique of the presently disclosed subject matter as a whole or a functional step thereof could be thus implemented by a remote network computer or a combination of several. Thus, any functional part of system 100 can be provided or connected via a computer network. In addition, the control unit can also remotely provide processor services over a network.

In one embodiment, a monitoring system for use with diabetic treatment management is provided such that it is deployed on a network computer such as a server which permits communication with users across the network. The monitoring system includes a communication interface configured and operable to permit access to stored raw log data obtained over a certain time and being indicative of glucose measurements, events and insulin delivery. The raw log data input can thus be communicated to the server over the network. This can take the form of uploading all or part of the raw log data input to the monitoring system.

Each such program may be stored on a storage medium or device, e.g., compact disc read only memory (CD-ROM), hard disk, magnetic diskette, or similar medium or device, that is readable by a general or special purpose programmable machine for configuring and operating the machine when the storage medium or device is read by the computer to perform the procedures described in this document. The system may also be implemented as a machine-readable storage medium, configured with a program, where the storage medium so configured causes a machine to operate in a specific and predefined manner.

The technique of the presently disclosed subject matter may use information from an existing third-party Diabetes Management System (DMS) such as a secured and HIPAA (Health Insurance Portability and Accountability Act)-compliant diabetes data management platform. The data input includes at least one of insulin delivery, glucose levels, food, physical activity, and any information available from a patient's devices. Following data collection (e.g. downloaded from the personal devices), the gathered information is analyzed by the control unit 102 to identify at least one of insulin requirements (total daily requirements and differences during the course of the day), glucose patterns, glucose trends, meal insulin requirements, insulin treatment patterns, carbohydrate consumption, etc. The efficacy of the patient's glucose control may be verified according to the known glucose goals recommended for example by the American Diabetes Association (ADA), or set individually. The data output may be displayed to the patient as a form of a report with recommendations defining how to set the insulin injections regimen. The data output depends on the source of input. The recommendations may be given directly to the patients and other caregivers via an application and/or a website. The recommendations may be sent to a cloud by the transceiver, to allow remote counsel capabilities.

The insulin injections treatment regimen may include a plan for the long acting insulin (type of insulin, amount and time (or times) of injections) and/or a plan for the short acting insulin (bolus) that includes either a (1) CR, CF and target or (2) fixed dose, CF and target, or (3) meal size estimation, CF and target or (4) SSI plan, depending on the bolus plan type. The data output may thus comprise a recommendation on the insulin dosage to both basal and bolus injection plans if it corresponds to the treatment type applied.

More specifically, if an SSI plan is used, the final recommendation may be created by deciding on the final bolus plan recommendation based on the used bolus plan (rather than the input bolus plan) and the final bolus insulin change that is based on the glucose levels (after integration). In case of inconsistent boluses per bucket (i.e. period of the day), a proper comment may be outputted. If there is a recommendation to change, the new plan may be outputted as the amount of insulin to change upon the used plan. If no change is provided, the current plan may be outputted, if such exists (without changes).

If a CR/CF plan is used, the final recommendation may be created by deciding on the final CR/CF plan recommendation based on the input plan and the final changes for the CR, CF and target (after integration). If there is a recommendation to change, the new plan is outputted for viewing by the user. The calculated changes may be in percent of change (POC), but they are usually not presented to the HCP (or patient) as POC. If no change was provided, the current plan may be outputted as is (without changes). The final basal plan recommendation may also be based on the used basal plan and the final basal injection change (after integration). In case of inconsistent basal injections or a basal plan not used, a proper comment may be outputted.

The control unit 102 can perform procedures and analysis without human supervision. In this connection, it should be understood that the control unit 102 can provide the insulin injections treatment plan from raw log data and from data manually input by a user (e.g. via touch pad or keypad).

In some embodiments, the system 100 is able to perform a data integration between the raw log glucose data corresponding to a glucose pattern averaged for a certain period of time of at least a plurality of days and the user data, including event data being based on user's input. In a specific and non-limiting example, control unit 102 is configured and operable to find times with Special Events (SE). Special Events (SE) can be defined as periods having rapid glucose increment that is most likely caused by carbohydrate intake. This can be based on both glucose sources: (i) a CGM or FGM and (ii) SMBG: in (i) when the slope between two close BG measurements is greater than a certain rate of glucose change; and (ii) when the slope in a certain time window surrounding the measurement is greater than a certain rate of glucose change and the length of the united steep slope measurements is greater than a certain duration. After having identified these steep ascent periods, these periods should be padded with moderate ascent time, both before and after, to determine the start and end times of the SE.

In some embodiments, the control unit 102 is thus configured and operable for receiving and processing the raw glucose log data and an existing insulin injections treatment plan, and automatically determining a recommended patient-specific insulin injections treatment plan. Therefore, in these cases, the monitoring system 100 can receive the existing insulin injections treatment plan (e.g. current basal plan) and decide on a change to that plan based on the glucose levels. Alternatively, the control unit 102 may be configured and operable to receive the user data (e.g. basal injection records) and to identify the existing patient-specific insulin injections treatment plan based on these records. Then the control unit 102 may recommend a change to this identified patient-specific insulin injections treatment plan.

The control unit 102 may thus identify the existing basal plan based on the patient basal injection records in case there are a plurality of days with basal records. This may be implemented by identifying, on such days, the common number of daily basal injections (1 or 2). In case of a single daily basal injection, the most common dose of the injections may be used as the existing basal plan value, and the most common rounded hour as the injection time. In case of two daily basal injections, the above procedure may be performed for each injection separately.

More specifically, as described above, in case the inputs for the control unit 102 include the patient's reported insulin records, control unit 102 can identify the patient basal and/or bolus plans (if the bolus plan is of SSI) based on these records. Also, control unit 102 may be configured for correlating between the raw log glucose data, insulin records and the existing patient-specific insulin injections treatment plan to validate the existing patient-specific insulin injections treatment plan. Control unit 102 can also notify if there is any discrepancy between the provided plan and the observed plan. In a specific and non-limiting example, in case the provided plan is a basal plan of 30 units, and the observed plan, based on the basal records in the analyzed period, is of 33 daily units, control unit 102 notifies that there is a discrepancy between the provided plan and the observed plan of 3 daily units. In other words, the control unit 102 may receive the basal plan as an input (=30U). In addition, if the basal records in the analysis period exist, and are consistent, but with a different amount, then the control unit 102 can update the basal plan to that consistent amount (for example, 33U). In addition, it may notify the HCP about this mismatch between the input plan and the “used” plan. The control unit 102 is capable of identifying this gap and to consider these 33 units as the “used” basal plan. The control unit 102 can notify that there was a gap between the provided plan and the “used” plan. If the basal records are inconsistent, the control unit 102 will not update the “used” plan, and instead will notify that it found inconsistency in the basal records. Alternatively or additionally, control unit 102 can identify whether the patient is using a fixed dose or SSI bolus plan different from the provided bolus plan.

In some embodiments, the monitoring system 100 of FIG. 1 may prepare a bolus event list based on the bolus records, that includes some informal data based on a per-bolus investigation. This information may be used for the plan analysis to determine if a bolus is valid to be used for the plan analysis (equivalent to having zero weight). The investigation tries to identify at least one of the following types of information for each bolus:
end event glucose value i.e. the minimal glucose that is found within a time frame depending on the bolus insulin type;
peak postprandial i.e. the maximal glucose that is found within a time frame after a bolus;
whether a bolus caused a hypoglycemia;
whether the bolus was given after a hypoglycemia;
whether the bolus that was given is priming;
whether the bolus is close to previous boluses;
whether the bolus has a timing issue relative to the meal timing;
whether the bolus is an outlier with respect to all boluses found in the same bucket—in case of SSI;
whether the bolus was overridden with respect to the estimated bolus as calculated by the current plan—in case of CR/CF;
the relevant bolus information (glucose level, carbs or estimated carbs, CR, CF, target, replayed amount of insulin)—in case of CR/CF;
whether the bolus was reported by a connected insulin pen, manually reported by the patient, or estimated by the system;
weight for each bolus based on all previous information (an invalid bolus will get zero weight and will not affect the insulin injections treatment plan).

The bolus may be considered invalid in at least one of the following cases: after hypo; the bolus has no end of event glucose; the bolus was given too late with respect to the carbohydrate intake; the bolus was given too close after another bolus; for SSI—the bolus is an outlier in its relevant bucket and/or for CR/CF-too big override.

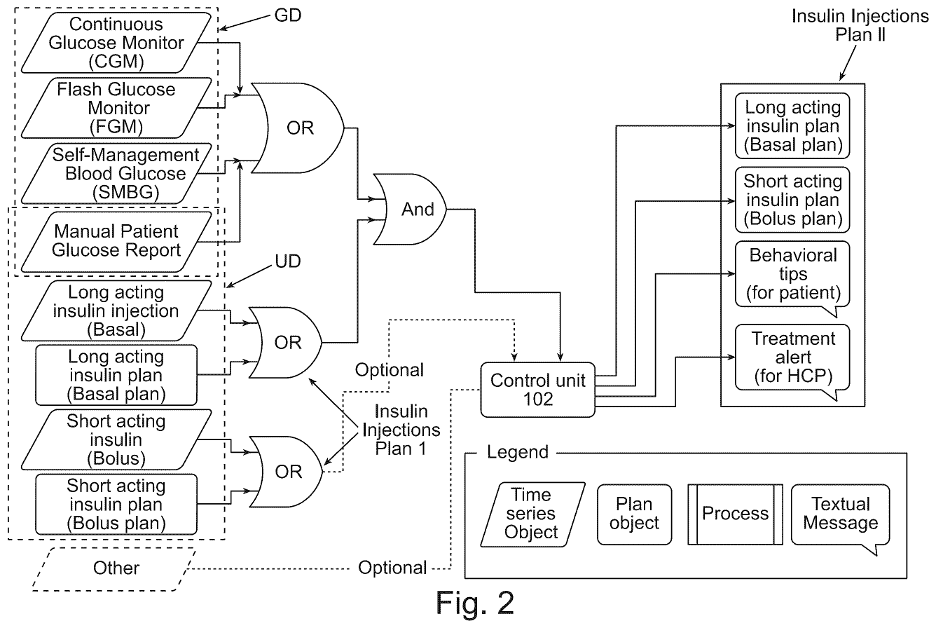

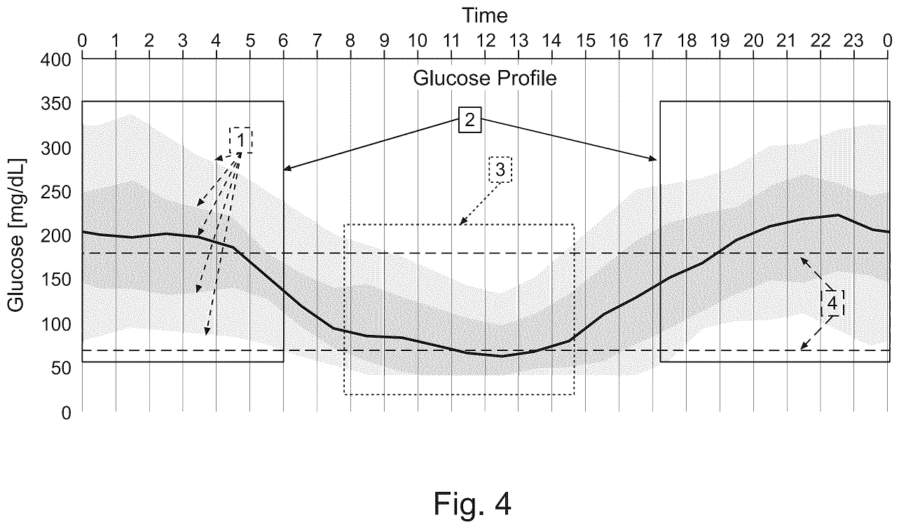

Reference is made to FIG. 2 exemplifying a schematic block diagram illustrating, in a non-limiting manner, the possible input(s) and output(s) of the monitoring system according to some embodiments of the presently disclosed subject matter. Control unit 102 receives inter alia raw log glucose data GD being indicative of at least one blood glucose pattern defining blood glucose levels over a certain time period. In general, the blood glucose pattern may be defined as a product of analyzing the raw glucose data over several days. To obtain a blood glucose pattern, the raw glucose data (date-time and value) can be processed as follows:
Divide hours of the day into segments. For example, 30 minutes segments: between 00:00-00:30, between 00:30-01:00, between 01:00-01:300 . . . etc.
On each day, collect the measurements found in each segment. For example, all measurements found in day 1 between 00:00-00:30.
For every such collected segment (per day), calculate the average glucose level. Relating to the example above, this average glucose level is the representative glucose level for day 1 between 00:00 and 00:30.
Now, assuming that X days are considered in the analysis, calculate the different percentiles across days for every segment of the day. This will be illustrated below with respect to FIG. 4.

As illustrated in FIG. 2, for example, raw log glucose data GD may comprise direct glucose measurement (i.e. measured data (including time and value) collected by a CGM, FGM or SMBG) or optionally user's input UD (i.e. glucose data collected by the patient over the certain time period and manually entered into the monitoring system, insulin injections or the basal and bolus plans). In combination with raw log glucose data GD, control unit 102 may also receive an existing patient-specific insulin injections treatment plan referred to in FIG. 2 as Insulin injections plan I for insulin delivery over the certain time period (e.g. long acting insulin injection). The Insulin injections plan I is an input plan for the Insulin injections treatment that may include individualized insulin dosing injection parameters data over the certain time period. The individualized insulin dosing injection parameters data comprises a basal plan (e.g. long acting insulin plan) and a bolus plan (e.g. short acting insulin plan). The user data UD may include raw data from the patient records, including the basal data and the bolus data. Basal data may include the time of the basal, the amount delivered, and the insulin type. Bolus data may include the time of the bolus, the amount delivered, carbohydrate input or meal estimation, and BG input and the insulin type. More specifically, if an SSI plan is applied, the bolus data includes at least one of day time, glucose range, amount and type of insulin, and if a CR/CF plan is applied, the bolus data includes time of day, glucose target, carbohydrates amount and. Other data such as information on the food components (e.g. fat) or physical activity may be received and analyzed by control unit 102. The term “BG input” refers to the glucose that was manually entered by the patient into the bolus event. Control unit 102 processes the received data and automatically determines a recommended patient-specific insulin injections treatment plan referred to in the figure as Insulin injections plan II. The Insulin injections II comprises individualized insulin dosing injection parameters data over the certain time period. The individualized insulin dosing injection parameters data comprises a basal plan (e.g. long acting insulin plan) and/or a bolus plan (e.g. short acting insulin plan) and/or personalized diabetes management tips including behavioral tips for a patient and/or a treatment alert for health care professionals (HCP). For example, the treatment alert of HCP may include warnings to alert the physician about gaps between the plans and the actual bolus/basal records, or for inconsistency in the records.

In some embodiments, control unit 102 is configured and operable to detect and alert for the most common behavioral tips for hypo and/or hyper glycaemia events (i.e. behavioral issues that caused hypo and hyper glycaemia). For each hyper glycaemia event, control unit 102 is configured and operable to find all possible behavioral reasons, such as untreated meals, bolus timing, or overtreating hypoglycemia. Similarly, for each hypo glycaemia event, control unit 102 is configured and operable to find all possible behavioral reasons, such as bolus timing or bolus stacking. From the reasons that were found to be valid (according to the number of occurrences and the rate of occurrences from all reasons), the ones with the highest priority may be identified and outputted as a behavioral tip. Therefore, in addition to personal insulin titration for the basal and bolus injections, control unit 102 can generate personal diabetes management tips. These tips are based on the glucose and/or insulin data.

Examples of tips provided for insulin injections patients may be in the form of textual notifications, as follows:
“I noticed that many of your highs may be avoided. Delivering an insulin bolus for every meal and snack may help you get better outcomes.
I noticed that many of your highs may be avoided. Delivering your bolus 15-20 minutes before eating may help you get better outcomes.
You are overtreating your lows. Eat moderately when treating your lows.
I noticed that many of your lows may be avoided. Remember to check your glucose level and bolus before you start eating.”

The monitoring system 100 is thus configured and operable to provide some advice for the patient's behavior and treatment settings, based on the patient's data. Moreover, the system is configured for recommending adjustments to the patient-specific treatment plan. As illustrated in FIG. 2, the insulin injections plan can be received directly from the insulin injections plan inputs, but in case of the basal and the SSI bolus plan, it can be automatically detected from the basal and bolus injection records, respectively. The insulin injections plan I can thus include (i) a basal injection plan (i.e. long acting insulin, even if a basal plan does not exist, or it does not correspond to the patient's actual basal records) and/or (ii) a bolus plan (in case of SSI, even if the bolus plan does not exist, or it does not correspond to the patient's actual bolus records), in one of the following ways: (1) CR and CF plan which may include recommendations for the CR and/or CF and/or the bolus target glucose levels; and/or (2) fixed dose, CF and glucose target and/or (3) meal size estimation and CF and/or (4) sliding scale (SSI) plan based on the bolus records and/or (iii) personalized diabetes management tips including behavioral tips to avoid periods of hyper and hypoglycemia such as timing of meal boluses, bolus delivery compliance and/or notes for the physician. By knowing the type of plan received in insulin injections plan I, the system is able to provide a new insulin injections plan II with the same type.

In a specific and non-limiting example, the sliding scale may be divided as presented in Table:

TABLE 1

| | Sliding Scale (SSI) | | |
|---|---|---|---|
| Type of bolus plan in | Measured glucose value | | |
| the form of: | From | To | Bolus amount |
| | 0 | 150 | 5 U |
| | 151 | 200 | 6 U |
| | 201 | 250 | 7 U |
| | 251 | 300 | 8 U |

FIG. 2 illustrates a specific and non-limiting example of the presently disclosed subject matter in which the personalized diabetes management tips are textual notifications provided to the patient or to health care professionals.

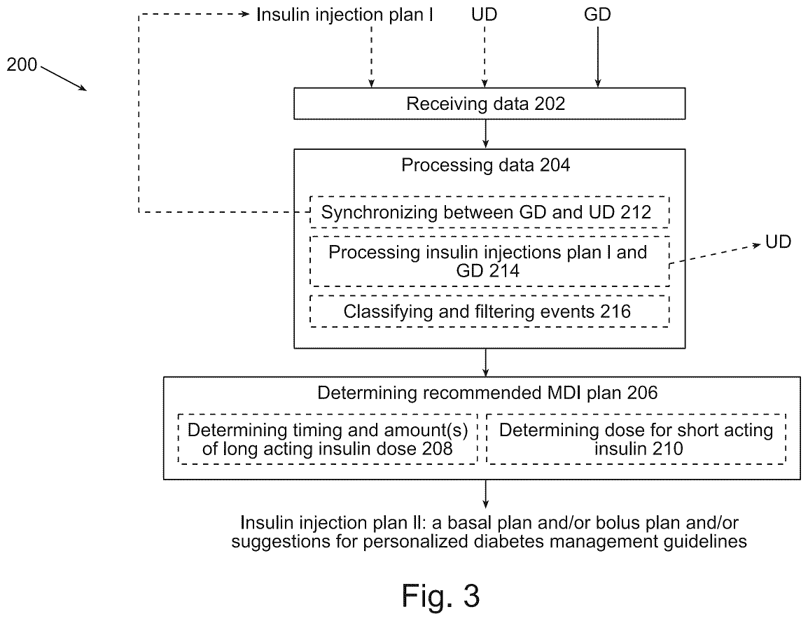

Reference is made to FIG. 3 exemplifying a possible method for use in monitoring the diabetic treatment of a diabetic patient based on insulin injections, according to some embodiments of the presently disclosed subject matter. Method 200 is aimed at automatically monitoring diabetes-related treatment. Method 200 comprises receiving raw log glucose data GD and (i) an existing patient-specific insulin injections treatment plan referred to in FIG. 3 as insulin injections plan I or (ii) user data in 202 referred in FIG. 3 as UD and processing the raw log glucose data GD with (i) the existing insulin injections treatment plan insulin injections plan I or (ii) the user data UD in 204, to automatically determine a recommended patient-specific insulin injections treatment plan in 206 referred to in FIG. 3 as insulin injections plan II. As described above, the raw log glucose data GD is indicative of at least one blood glucose pattern defining blood glucose levels over a certain time period. The user data UD comprises data being indicative of insulin delivery over the certain time period and/or data being indicative of meals consumed during the certain time period.

As described above, determining the recommended patient-specific insulin injections treatment plan insulin injections plan II comprises determining individualized insulin dosing injection parameters data over the certain time period. The individualized insulin dosing injection parameters data comprises a basal plan and/or a bolus plan and/or suggestions for personalized diabetes management tips. The recommended patient-specific insulin injections treatment plan (insulin injections plan II) comprises an insulin treatment plan which may include all types of bolus plans such as CR/CF, SSI, fixed dose+CF or meal size estimation+CF, compliant with insulin injections plan I. Determine a recommended patient-specific insulin injections treatment plan in 206 may comprise automatically determining a timing and amount(s) of long acting insulin dose in 208 and/or determining a dose of short acting insulin in 210. More generally, determining the insulin treatment plan in 206 may comprise determining at least one of timing of insulin delivery an amount of bolus dosage, an amount of long acting insulin dosage, number of required doses of basal insulin, CR according to the time of day, and CF, or individual bolus target. Determining an amount of long acting insulin dose in 208 may comprise analyzing the glucose pattern of the GD to define a certain time of injecting the long acting insulin dose and a certain number of required doses of the basal insulin. Determining short acting insulin dose for meals in 210 may comprise determining a short-acting insulin dosage component taken according to a sliding scale.

Reference is made now to FIG. 4 illustrating an example of a processed glucose pattern allowing providing a recommendation for changing the insulin treatment plan according to some embodiments of the presently disclosed subject matter. A raw data may be collected during X days and different glucose percentiles may be calculated across the X days for every segment of the day. The different glucose percentiles are represented as arrow 1 showing that the light area shows the 10^{th} and 90^{th} quantile range, the darker area is the 25^{th} and 75^{th} quantile range, and the line is the 50^{th} percentile (median). After having calculated these percentiles, the technique can identify period(s) of time during the day being characterized by a pattern of high glucose levels, and can mark this period as high glucose pattern (Arrow 2 showing rectangle in FIG. 4). Or alternatively, the technique can identify period(s) of time during the day being characterized by a pattern of low glucose levels along with the presence of hypoglycemia events, and therefore can mark this period as low glucose pattern (Arrow 3 showing rectangle in FIG. 4). In other words, the pattern of low glucose levels is defined by the percentiles of the glucose pattern, but also requires identifying the presence of hypoglycemia events in this period (i.e. segment) of the day. Arrows 4 mark the lines showing the consensus target range of 70 and 180 mg/dL.

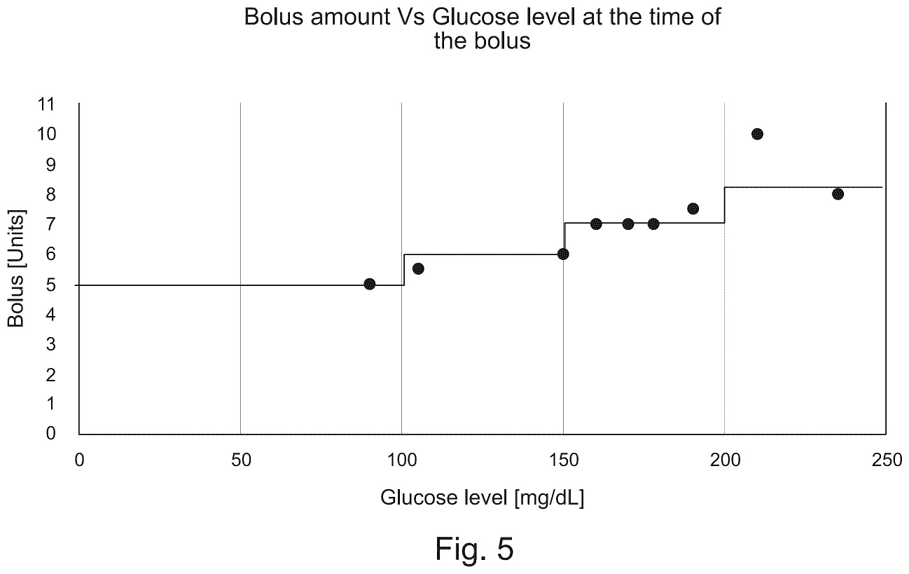

Reference is made to FIG. 5, illustrating an example for the patient's provided morning bolus plan (as detailed in Table 2 below) and the amount of insulin per bolus, per glucose. FIG. 5 shows a plot of boluses amount given for a specific glucose level as reported by the patient. The raw log glucose data includes the glucose at the time of the bolus which enables to estimate the bolus plan. The raw log glucose data can be taken from the glucose level as entered by the patient (e.g. to a log book), or by correlating the glucose level found at the time of the bolus in the patient's CGM or other glucose device.

TABLE 2

| From | | Amount |
|---|---|---|
| (mg/dL) | To (mg/dL) | (Units) |
| 0 | 100 | 6 |
| 101 | 150 | 7 |
| 151 | 200 | 8 |
| 200 | 250 | 9 |
| 250 | High | 10 |

As can be seen in FIG. 5, the amount of insulin per bolus is smaller than the amount of insulin shown in the bolus plan in Table 2. The actual bolus plan that the control unit 102 can identify is shown in Table 3 below. In addition, the control unit 102 is capable of notifying that a discrepancy was found between the reported boluses and the provided bolus plan in the morning time. Table 3 shows an example of a patient's SSI bolus plan being determined by using the patient's reported bolus records.

TABLE 3

| From | | Amount |
|---|---|---|
| (mg/dL) | To (mg/dL) | (Units) |
| 0 | 100 | 5 |
| 101 | 150 | 6 |
| 151 | 200 | 7 |
| 200 | 250 | 8 |
| 250 | High | 9 |

The control unit 102 may also identify the existing bolus plan for every defined time window (e.g. morning, afternoon, evening). This may be implemented by defining a common bolus step, defining if the boluses are consistent; taking out outlier boluses and boluses with no corresponding glucose value; estimating the SSI plan based on the left boluses in case of no bolus plan or use the bolus plan and calculate the distance of the boluses from the current plan. The system 100 may also estimate the required bolus change for each period/bucket of the day (morning/afternoon/evening etc.).

For each bolus, the needed insulin change may be calculated, and then,
In case of SSI—only if there are more than a certain minimal number of boluses, defining the direction of change that is dominant in each bucket. The number of the minimal boluses required for estimating the bolus plan can change based on the bolus records. According to the dominant direction, calculating the plan change as the weighted average of the bolus change decrease/increase and rounded down/up according to the insulin pen resolution. It should be also noted that the bolus plan can change also without any bolus records.
In case of CR/CF—using the calculated change to estimate the changes for the CR and CF separately. This can be done by using the insulin for meal ratio as a threshold to determine whether the bolus can be used to calculate the CR or CF, and also to determine the weight for each bolus. Only in cases of more than a certain number of boluses, and a significant weight in either direction, the system may suggest a plan change (in the significant direction). It should be also noted that the bolus plan can change also without any bolus records.

In some embodiments, control unit 102 estimates times of delivered boluses based on the patient's continuous glucose levels. Utilizing the glucose levels and duration of glucose gradients, control unit 102 defines the times of the estimated boluses and also classifies them into three different types: carbohydrate consumption only, correction of glucose levels only, or a mix of the two.

In some embodiments, control unit 102 is configured and operable to process the raw log glucose data to thereby identify, in the raw log glucose data, event data being indicative of at least one of insulin delivery or meal(s) consumed in a certain time period; classifying the event data in different events relating or not to the existing patient-specific insulin injections treatment plan, and filtering out the events not relating to the existing patient-specific insulin injections treatment plan. The filtered events are those that might have had a potential misbehavior on the user's part, for example if the patient forgot to deliver a bolus when he started to eat, and, instead, he delivered it an hour later.

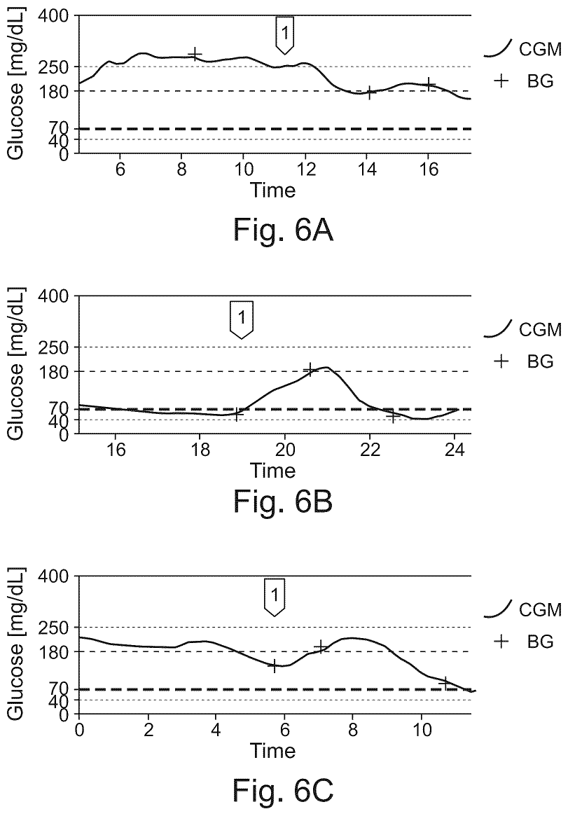

Reference is made to FIGS. 6A-6C illustrating examples of automated determination of bolus delivery based on continuous glucose levels. More specifically, FIG. 6A shows an estimated bolus classified as a correction bolus. (1) in FIG. 6A indicates the estimated bolus time for correcting the blood glucose. The estimated bolus time for correcting the blood glucose was determined by identifying a steep decrease in the glucose levels with minimal duration, not following SE. Then, in some cases, linking between the glucose decrement and the SMBG measurement enables to better estimate the bolus time. FIG. 6B shows an estimated bolus classified as a meal bolus. (1) in FIG. 6B indicates the estimated bolus time for treating carbohydrate intake. The estimated bolus classified as a meal bolus was determined by identifying a steep decrease in the glucose levels with minimal duration, following SE with steep slope. FIG. 6C shows an estimated bolus classified as a mixed bolus. (1) in FIG. 6C indicates the estimated bolus time for treating both carbohydrate intake and high glucose levels, by finding the same event as described above for meal bolus and correlating it to high glucose measurement (SMBG/CGM/FGM or manually reported) at the time of the bolus.

In some embodiments, the insulin plan changes may be based on the glucose levels (from either measurement device or the manual entries of glucose levels by the patient), insulin records (from either a connected pen and/or manual entered by the patient), insulin injections treatment plan in this case CR/CF plan (Table 4A and Table 4C) and additional information that was entered by the patient (i.e. carbohydrates amount, physical activity and more).

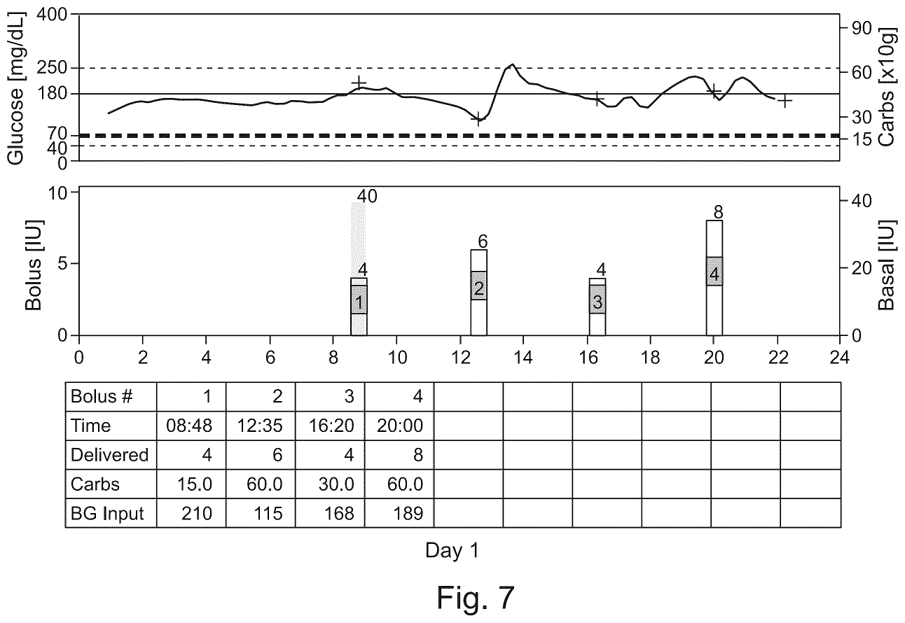

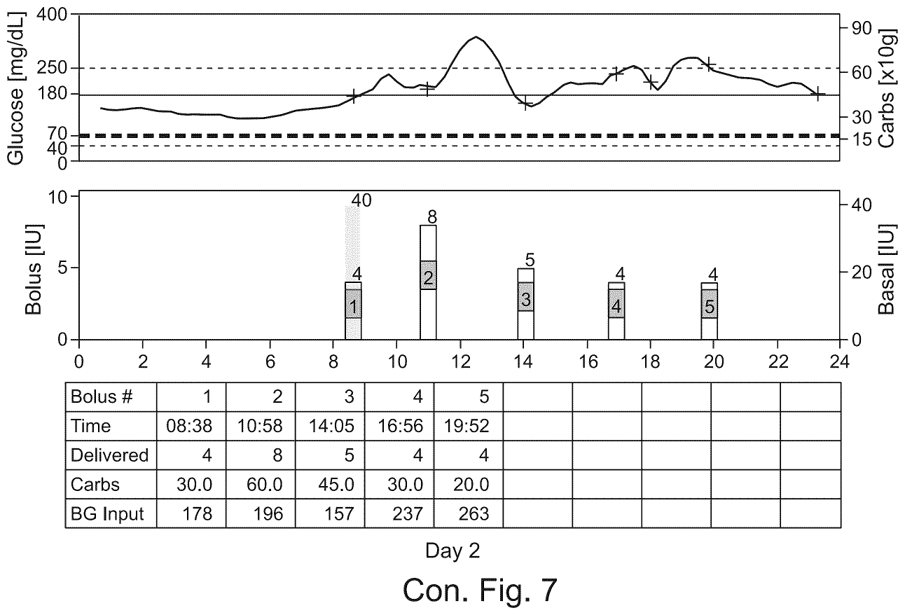

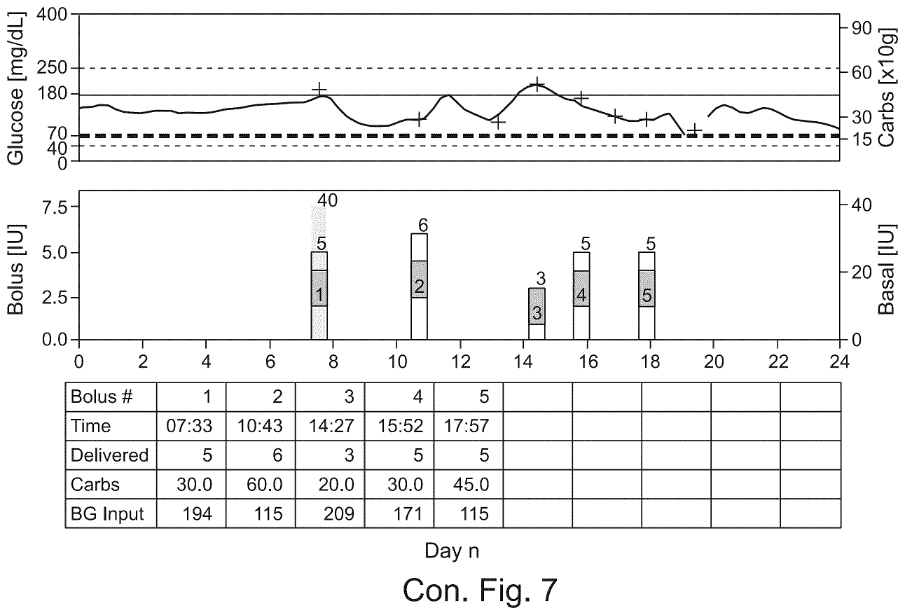

Reference is made to FIG. 7 illustrating an example of such input data (GD and UD), including a day-by-day view. Each day includes the glucose levels (upper panel), the reported insulin injections (lower panel) that includes the basal and bolus along with table that provides some details for every bolus (i.e., the bolus time, amount delivered, carbohydrates and BG as was reported by the patient). The recommended insulin injections treatment plan based on these input data can be seen in Table 4B and Table 4D. In some embodiments, the control unit is configured and operable for automatically determining an amount of long acting insulin dose by analyzing the blood glucose pattern to define a certain time of injecting the long acting insulin dose and a certain number of required doses of the basal insulin. More specifically, in this specific and non-limiting example, the system of the presently disclosed subject matter received the raw log data and recommended to split the long acting insulin injection into two separate injections (Table 4A and Table 4B).

TABLE 4A
Current basal plan

| Time | Amount |
|---|---|
| 08:00 | 40 Units |

TABLE 4B
Recommended basal plan

| Time | Amount |
|---|---|
| 08:00 | 20 Units |
| 20:00 | 18 Units |

TABLE 4C
Current bolus plan

| | Morning | Afternoon | Evening | Bedtime |
|---|---|---|---|---|
| | (05:00-11:00) | (11:00-17:00) | (17:00-22:00) | (22:00-05:00) |
| Carbs ratio | 12 | 10 | 10 | 12 |
| (g/U) | | | | |
| Correction | 60 | 50 | 50 | 60 |
| factor | | | | |
| (mg/dL/U) | | | | |
| Blood | 140 | 140 | 140 | 140 |
| Glucose target | | | | |
| (mg/dL) | | | | |

TABLE 4D
Recommended bolus plan

| | Morning | Afternoon | Evening | Bedtime |
|---|---|---|---|---|
| | (05:00-11:00) | (11:00-17:00) | (17:00-22:00) | (22:00-05:00) |
| Carbs ratio | 12 | 10 | 10 | 14 |
| (g/U) | | | | |
| Correction | 60 | 50 | 50 | 60 |
| factor | | | | |
| (mg/dL/U) | | | | |
| Blood | 140 | 140 | 140 | 140 |
| Glucose target | | | | |
| (mg/dL) | | | | |

In some embodiments, the insulin plan changes may be based on the glucose levels with no insulin records. The observed changes are based on the pattern that is detected in the patient glucose profile. Therefore, the technique of the presently disclosed subject matter can provide recommendation for changing the insulin treatment plans even in case of no basal and/or bolus records. In these cases, the control unit considers the estimated boluses and their classification as described above. In case not enough estimated boluses are found, the control unit can recommend changes based on the glucose patterns alone.

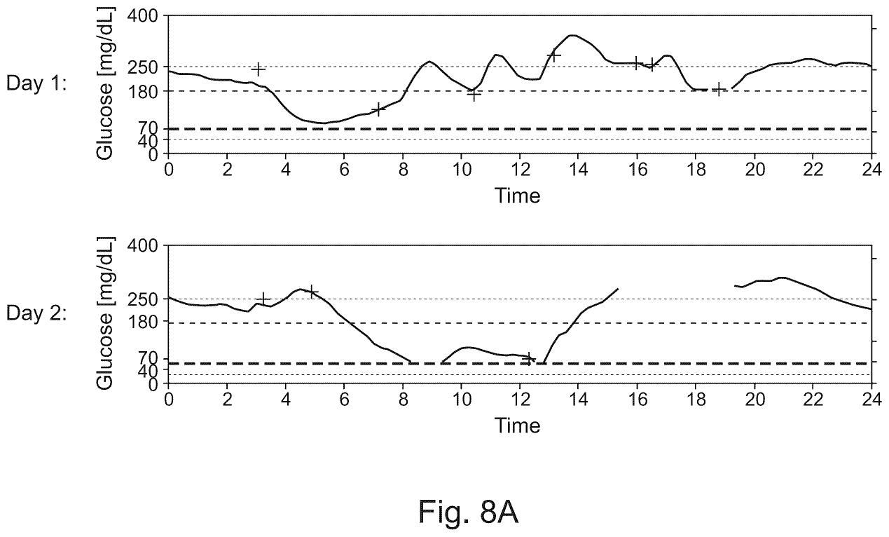

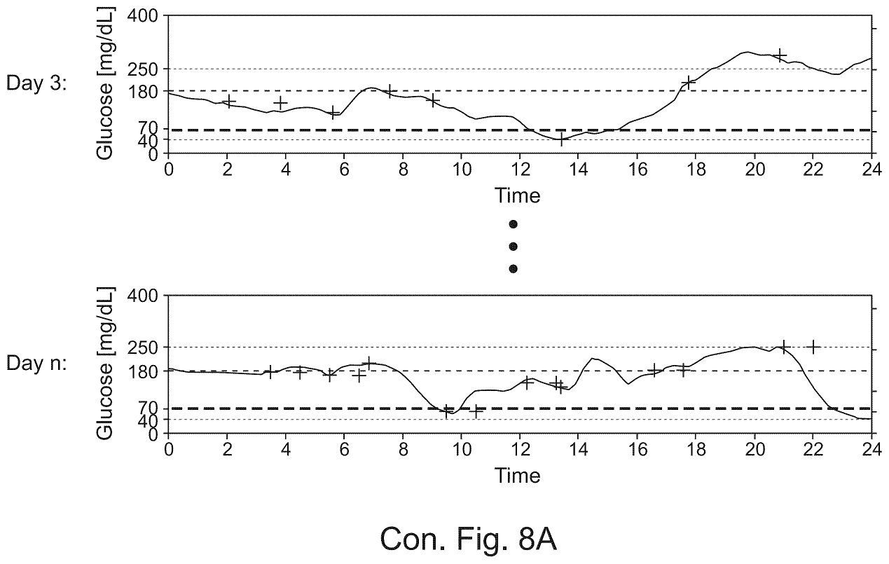

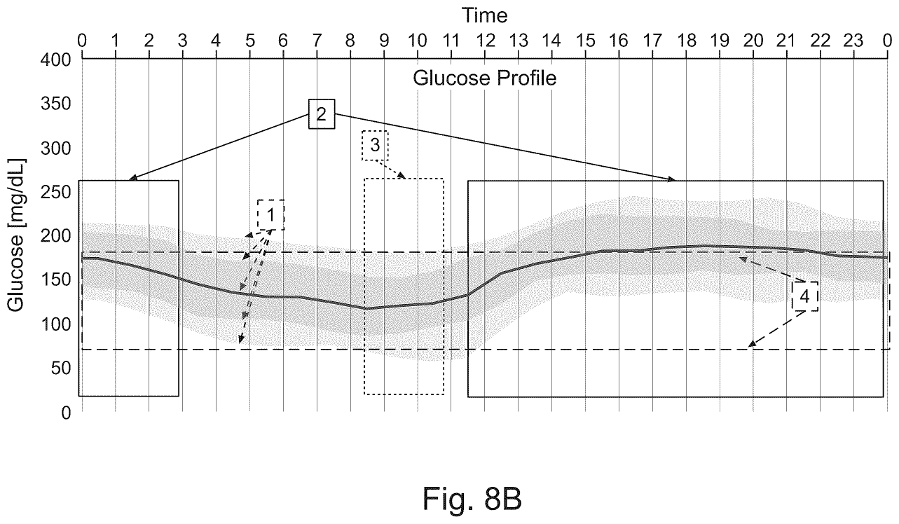

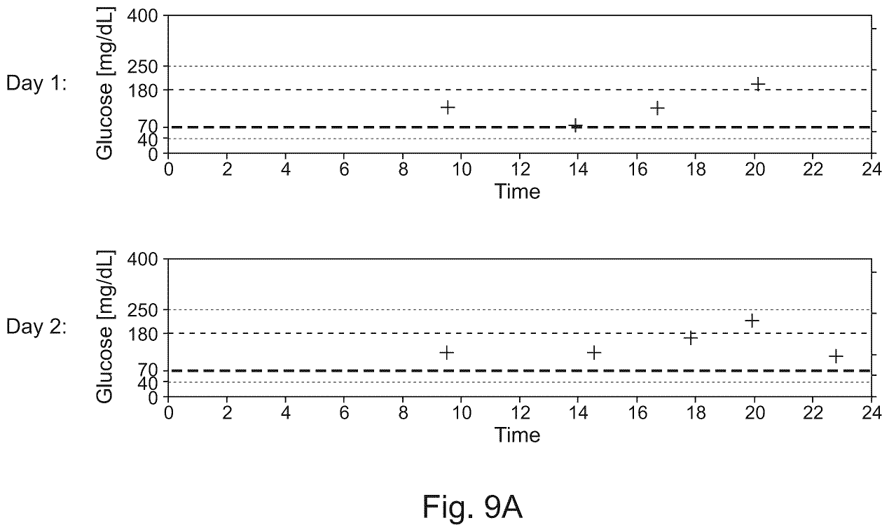

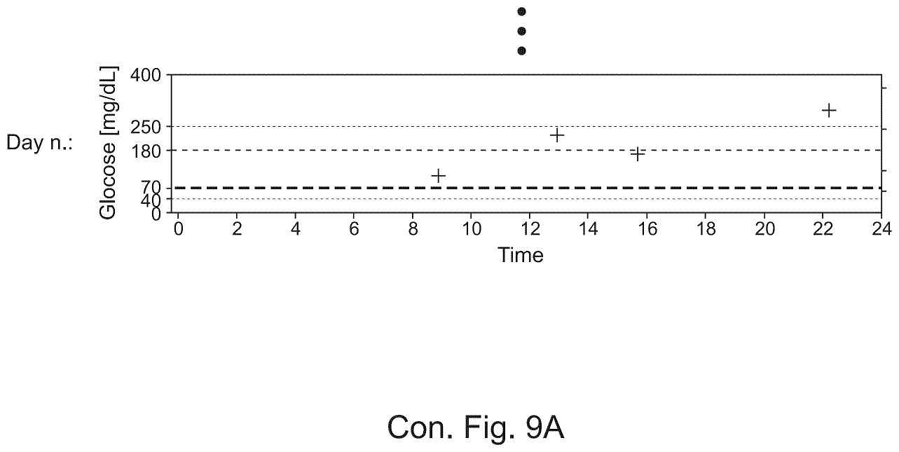

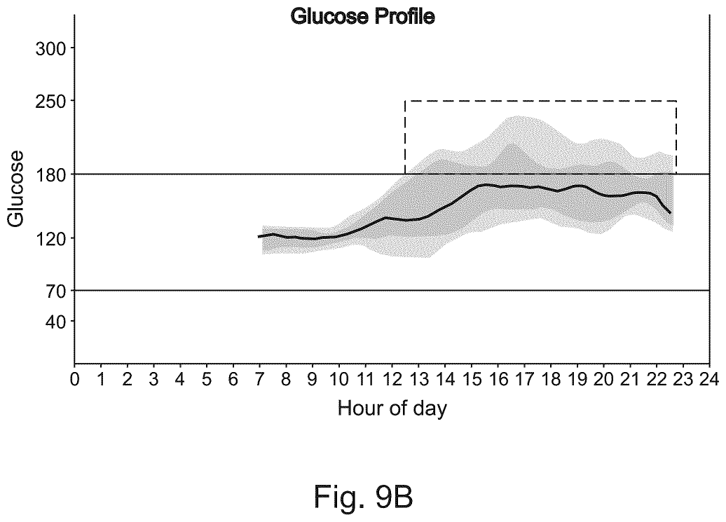

Reference is made to FIGS. 8A-8B and FIGS. 9A-9B illustrating an example of change recommendations based on glucose data only. More specifically, FIG. 8B shows an example of a glucose pattern that was created from the CGM raw data (FIG. 8A) along with a complete recommendation plan for both the basal and the bolus (carb counting type). FIG. 8A presents the daily glucose data, where the black line is the glucose values of the CGM, and the crosses are the SMBG values. In FIG. 8B, arrows (1) refer to glucose percentiles, where the light area is the 10th and 90th range, the darker area is the 25th and 75th quantile range and the line is the 50th quantile. Arrows (2) show two time periods where the system detected a pattern of high glucose levels. Arrow (3) shows a time period where the system detected a pattern of low glucose levels.

Table 5A and Table 5D below present the inputted insulin injections treatment plans along with a complete recommended treatment plan (Table 5B and Table 5C). More specifically, in this specific and non-limiting example, the system of the presently disclosed subject matter received the raw log data and identified low fasting glucose levels (i.e. the first glucose levels found after wake-up) which resulted in basal decrement (Table 5A and Table 5B). The high glucose levels found from the late morning till early morning result in an increasing the amount of insulin for the boluses (by reducing the CR and CF (Table 5C and Table 5D)).

### Basal Plan

TABLE 5A
Current plan

| Time | Amount |
|---|---|
| 08:00 | 25 Units |

TABLE 5B
Recommended plan

| Time | Amount |
|---|---|
| 08:00 | 23 Units |

### Bolus Plan

TABLE 5C
Recommended bolus plan

| | Morning | Afternoon | Evening | Bedtime |
|---|---|---|---|---|
| | (05:00-11:00) | (11:00-17:00) | (17:00-22:00) | (22:00-05:00) |
| Carbs ratio | 8 | 8 | 9 | 9 |
| (g/U) | | | | |
| Correction | 30 | 30 | 30 | 30 |
| factor | | | | |
| (mg/dL/U) | | | | |
| Blood | 110 | 110 | 110 | 110 |
| Glucose | | | | |
| target | | | | |
| (mg/dL) | | | | |

TABLE 5D
Current bolus plan

| | Morning | Afternoon | Evening | Bedtime |
|---|---|---|---|---|
| | (05:00-11:00) | (11:00-17:00) | (17:00-22:00) | (22:00-05:00) |
| Carbs ratio | 9 | 9 | 9 | 9 |
| (g/U) | | | | |
| Correction | 40 | 40 | 40 | 40 |
| factor | | | | |
| (mg/dL/U) | | | | |
| Blood | 110 | 110 | 110 | 110 |
| Glucose | | | | |
| target | | | | |
| (mg/dL) | | | | |

Table 6A and 6B below presents another example, focusing on the bolus treatment plan. More specifically, in this specific and non-limiting example, the system of the presently disclosed subject matter received the same raw log data, as presented in FIG. 8A and FIG. 8B, but with a bolus treatment plan that includes day and night segments only (Table 6B), instead of the morning, afternoon, evening and night. The recommended bolus plan (Table 6A) includes plans that are separated for the morning and night, without for example, splitting the day segment into: morning, afternoon, and evening.

### Bolus Plan

TABLE 6A
Recommended bolus plan

| | Morning | Night |
|---|---|---|
| | (05:00-22:00) | (22:00-05:00) |
| Carbs ratio (g/U) | 8 | 9 |
| Correction | 30 | 30 |
| factor (mg/dL/U) | | |
| Blood Glucose | 110 | 110 |
| target (mg/dL) | | |

TABLE 6B
Current bolus plan

| | Day | Night |
|---|---|---|
| | (05:00-22:00) | (22:00-05:00) |
| Carbs ratio (g/U) | 9 | 9 |
| Correction | 40 | 40 |
| factor (mg/dL/U) | | |
| Blood Glucose | 110 | 110 |
| target (mg/dL) | | |

FIGS. 9A-9B illustrate another example of automated determination of changes to the insulin injections treatment plan based on glucose levels, only based on SMBG data. More specifically, FIG. 9B shows an example of a glucose pattern that was created from the SMBG raw data (FIG. 9A) along with a complete recommendation plan for both the basal and the bolus of SSI type. However, it should be noted that the presently disclosed subject matter is not limited to the determination of a bolus of an SSI type. Using SMBG data only, the system can also change the basal plan or other types of bolus plans (CR/CF, Fix dose and CF or meal estimation and CF). FIG. 9A presents the daily glucose data, where the crosses are the SMBG values. In FIG. 9B, the percentiles are as described above.

Table 7A, 7C, 7E, 7G and 7I below present the inputted insulin injections treatment plans along with a complete recommended treatment plan (Table 7B, 7D, 7F, 7H and 7J). More specifically, in this specific and non-limiting example, the system of the presently disclosed subject matter received the raw log data and identified fasting glucose levels that are in the normal range, which resulted in maintaining the basal plan as is (Table 7A and Table 7B). The high glucose levels found from the afternoon till early late night, result in increasing the amount of insulin for the boluses at that time, and no changes for the morning and night time (Table 7C till Table 7J).

### Basal Plan

TABLE 7A
Current plan

| Time | Amount |
|---|---|
| 08:00 | 9 Units |

TABLE 7B
Recommended plan

| Time | Amount |
|---|---|
| 08:00 | 9 Units |

### Bolus Plan

TABLE 76C
Current plan
Morning (5 AM-11 AM)

| From | To | Amount |
|---|---|---|
| (mg/dL) | (mg/dL) | (Units) |
| 0 | 100 | 3 |
| 101 | 150 | 4 |
| 151 | 200 | 5 |
| 200 | 250 | 6 |
| 300 | High | 7 |

TABLE 7D
Recommended plan

| From | To | Amount |
|---|---|---|
| (mg/dL) | (mg/dL) | (Units) |
| 0 | 100 | 3 |
| 101 | 150 | 4 |
| 151 | 200 | 5 |
| 200 | 250 | 6 |
| 300 | High | 7 |

TABLE 7E
Afternoon (11 AM-5 PM)

| From | To | Amount |
|---|---|---|
| (mg/dL) | (mg/dL) | (Units) |
| 0 | 100 | 3 |
| 101 | 150 | 4 |
| 151 | 200 | 5 |
| 200 | 250 | 6 |
| 300 | High | 7 |

TABLE 7F

| From | To | Amount |
|---|---|---|
| (mg/dL) | (mg/dL) | (Units) |
| 0 | 100 | 4 |
| 101 | 150 | 5 |
| 151 | 200 | 6 |
| 200 | 250 | 7 |
| 300 | High | 8 |

TABLE 7G
Evening (5 PM-10 PM)

| From | To | Amount |
|---|---|---|
| (mg/dL) | (mg/dL) | (Units) |
| 0 | 100 | 3 |
| 101 | 150 | 4 |
| 151 | 200 | 5 |
| 200 | 250 | 6 |
| 300 | High | 7 |

TABLE 67H

| From | To | Amount |
|---|---|---|
| (mg/dL) | (mg/dL) | (Units) |
| 0 | 100 | 4 |
| 101 | 150 | 5 |
| 151 | 200 | 6 |
| 200 | 250 | 7 |
| 300 | High | 8 |

TABLE 7I
Night (10 PM-5 AM)

| From | To | Amount |
|---|---|---|
| (mg/dL) | (mg/dL) | (Units) |
| 0 | 100 | 3 |
| 101 | 150 | 4 |
| 151 | 200 | 5 |
| 200 | 250 | 6 |
| 300 | High | 7 |

TABLE 7J

| From | To | Amount |
|---|---|---|
| (mg/dL) | (mg/dL) | (Units) |
| 0 | 100 | 3 |
| 101 | 150 | 4 |
| 151 | 200 | 5 |
| 200 | 250 | 6 |
| 300 | High | 7 |

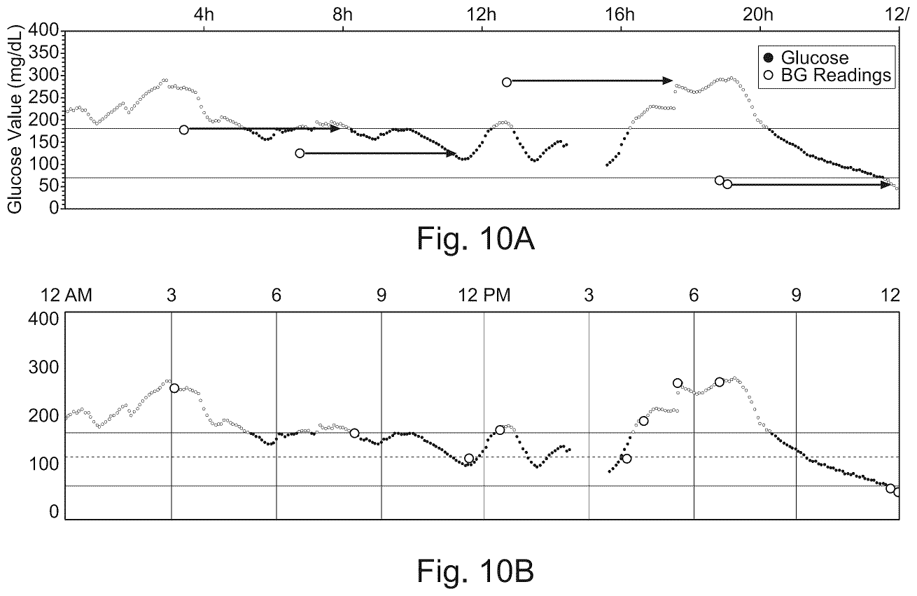

In some embodiments, processing the raw log glucose data GD in 204 may comprise synchronizing between the measured data generated by the plurality of external devices and/or between the raw log glucose data GD and the user data UD in 212 as illustrated for example in FIGS. 10A-10B. FIGS. 10A-10B graphically illustrate an example in which two sources of glucose data are synchronized according to some embodiments of the presently disclosed subject matter. The system is configured to identify cases where there is a clear asynchronization between the measurement devices, and to synchronize them. The synchronization process enables to align between the different source data (not limited to a specific number of data sources). In this specific and non-limiting example, FIGS. 10A-10B present such a synchronization between SMBG and CGM data. In particular, FIG. 10A presents the raw glucose data, where the single dots are the SMBG measurement, and the smaller dots, forming curved shapes, are the CGM data before synchronization. The arrow represents the shift in time that was detected between these two devices. FIG. 10B shows the result of that synchronization process by using the teachings of the presently disclosed subject matter. As can be seen, the SMBG measurements are now perfectly aligned with the CGM.

In some embodiments, processing the raw log glucose data GD in 204 may comprise receiving and processing the raw log glucose data GD and to identify the user data UD in 214. Moreover, event data being indicative of at least one of insulin delivery or meal(s) consumed in a certain time period may be identified in the raw log glucose data. The event data may then be classified in different events relating or not to the existing patient-specific insulin injections treatment plan, and the events not relating to the existing patient-specific insulin injections treatment plan may be filtered out in 216.

Generally, method 200 is not targeted to change the insulin type, neither for the basal, nor the bolus injections. Nevertheless, in case of sufficient basal/bolus records, the system can recommend a treatment plan based on the injection records even if it is inconsistent with the basal/bolus plans, respectively. The system is thus capable of identifying the inconsistence or discrepancy between the basal/bolus plans and the basal/bolus records. Moreover, the technique of the presently disclosed subject matter can identify if the patient needs to change from one to two daily injections or vice versa if the patient glucose levels indicate a need to add or reduce a basal injection.

In some embodiments, the method comprises identifying a start point and an end point of a certain inactive time period being indicative of an absence of insulin delivery and meal consumption. For example, for each day, the method may comprise identifying the fasting glucose i.e. the morning glucose value that represents the fasting glucose value. Moreover, the technique is capable of identifying the times where the glucose levels are most likely to be affected by the active insulin from the long acting insulin levels, and not by other sources of interference, such as carbohydrate consumption or other boluses. The glucose values at these times are used to analyze the patient basal doses.

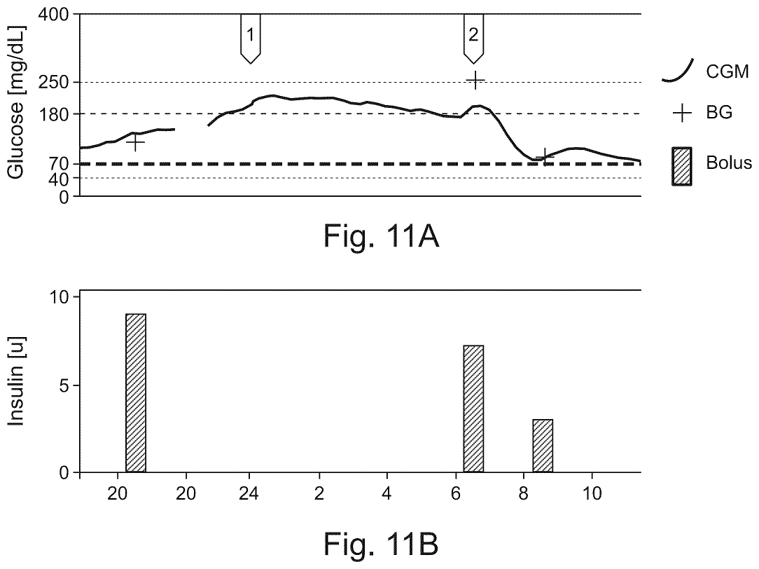

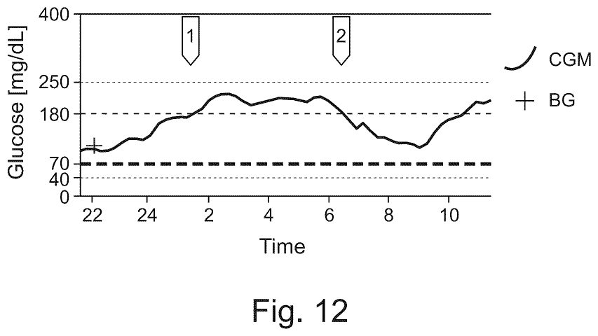

Reference is made to FIGS. 11A, 11B and 12 illustrating a first example in which both bolus records and glucose levels are used (FIGS. 10A-10B), and a second example in which only the glucose levels are used to determine the fasting period (FIG. 12). More specifically, FIG. 11A shows a real glucose data around the nighttime and FIG. 11B shows an insulin data around the nighttime. The technique determines that in FIG. 11A, (1) is the start time and (2) the end time of the fasting period because of the existence or absence of active insulin coming from boluses and/or record of meals and/or detected SE and/or existence of SMBG measurement. FIG. 12 shows a real glucose data around the nighttime. The technique determines that in FIG. 12, (1) is the start time and (2) the end time of the fasting period because the technique identified a meal event (SE) ending at around 2 AM and then a bolus (estimated bolus) at around 6 AM which ends the fasting period.

In a specific and non-limiting example, the default end time of the fasting period may be defined in the range of about 4 AM to 12 PM. However, it should be noted that these times are not limiting at all and that the exact time range can be set after accounting for glucose or patient interruptions to the fasting state, such as boluses, meals, hypoglycemia events, etc. The start time can be adjusted by the closest bolus time+some buffer or closest mealtime+some buffer, both of which are found before the fasting time per day. Then, the time of the patient action inside this adjusted time frame is identified, and the closest glucose is matched at this time or before (but not earlier than the fasting start time). The patient action can be a SMBG measurement, bolus injection record and/or basal injection record and/or detected SE and/or reported meal and/or hypoglycemia event. If no patient action was found, the average of all sensor data found within a certain time period may be calculated. If any hypo event, meal, bolus or steep descent up to certain number of hours to the fasting was identified, the measurement is then invalidated.

In some embodiments, the method may also comprise identifying, for each day, a pre-fasting glucose i.e. when a fasting glucose exists, the glucose value that represents the pre-fasting glucose. This value is the first value that is found after the latest interruption that is found between a certain time (e.g. 8 PM) till the fasting time on the following day. An interruption can be either: hypo, bolus, special event or descent event. Then, the pre-fasting time may be defined as the time of the interruption+buffer (depending on the interruption), but not after the fasting time minus some buffer. Then, the pre-fasting glucose value should be set as the average value found in the hour immediately after the pre-fasting time. In the following cases, this pre-fasting glucose value should be disregarded: 1) in case the time period between the fasting and pre-fasting is less than a certain minimum time period between the pre and post fasting, 2) in case of hypo events that started between the pre-fasting and fasting time, 3) no glucose is far enough from the fasting time.

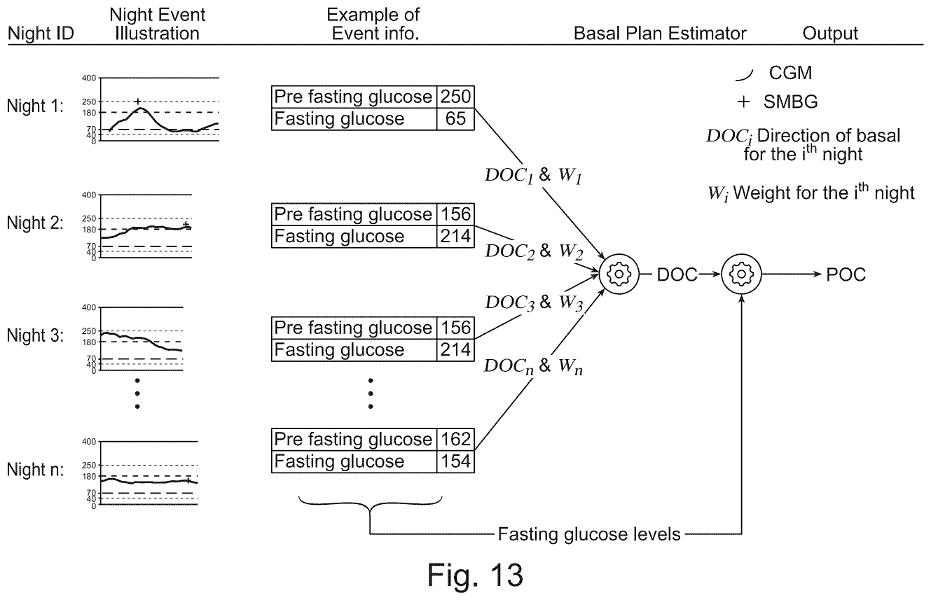

In some embodiments, the method is configured and operable to estimate the basal injection change based on the overnight glucose levels and the observed common basal dose. It integrates between the pre-fasting and fasting glucose if applicable where days with just fasting glucose levels get lower weight than days that have also pre-fasting glucose. Days with existing pre-fasting glucose levels within a range get the higher weight. For each day, the method determines if the basal injection should have been increased/stable, or decreased, based on the difference between the pre-fasting, fasting glucose (slope-algorithm) and the fasting glucose levels. Finally, the method counts the number of increase/stable/decrease days, with respect to their weights, and decides on the direction of change. Then, based on that direction of change and the fasting glucose levels, it outputs a single recommendation for the basal injection. Reference is made to FIG. 13 illustrating a specific example of a possible internal analysis for a basal plan based on the pre-fasting and fasting glucose levels found in the pre-process phase. In this example, the control unit is configured to collect the raw glucose log data (i.e. glucose levels) and to determine the direction of change for the basal injection and the weight of that event for each night. In some cases, the weight for the night can be zero (for example: night 1). In this example the zero weight is due to the fact that there was a bolus event during the nighttime which affected the fasting glucose levels.

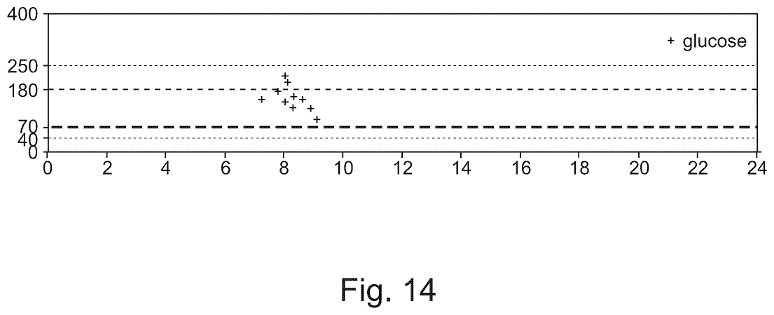

Reference is made to FIG. 14 illustrating an example in which only basal injection is used, referred as basal only treatment regimen. More specifically, FIG. 14 shows a real glucose data around the morning, signed as fasting glucose by the system. Table 7A below present the current basal injection plan, and Table 7B shows the recommended basal injection plan that was generated based on these fasting glucose levels.

TABLE 7A-7B

| Current basal plan | | Recommended basal plan | |
|---|---|---|---|
| Time | Amount | Time | Amount |
| 08:00 | 16 Units | 08:00 | 16 Units |

The following are examples for the patient-specific insulin injections plan based on the raw data and the recommendations for changes that the system can generate.

The basal plan can include one or two daily injections, and the system may in some cases recommend changes to daily dose and/or recommend changes to the number of daily basal injections (only if the basal records are present).

As for the bolus plan, there are four main types of bolus plans that are being used: one is a fixed amount of insulin for meals plus a kind of scaling to correct the glucose levels. Second is using carb counting and a correction factor to calculate the bolus amount (in a similar way to the insulin pump). Third, is using meal size estimation (for example small, medium and large) with a correction factor to correct the glucose values above some glucose target and fourth, is using fixed amount of insulin for meals plus a correction factor to calculate the bolus amount.

### Basal Plan

### Example I is Shown in Tables 8A-8B Below

A single basal daily injection is received and transformed into a single daily basal injection plan. The single basal daily injection can be inputted by the user or may also be determined from the basal records. The type of insulin can be of any type such as Lantus or Tregludec.

TABLE 8A-8B

| Current basal plan | | Recommended basal plan | |
|---|---|---|---|
| Time | Amount | Time | Amount |
| 08:00 | 20 Units | 08:00 | 22 Units |

### Example II is Shown in Tables 9A-9B Below

Two daily injections are received and transformed into two daily basal injections. The two daily injections can be inputted by the user or may also be determined from the basal records.

TABLE 10A-10B

| Current basal plan | | Recommended basal plan | |
|---|---|---|---|
| Time | Amount | Time | Amount |
| 08:00 | 20 Units | 08:00 | 18 Units |
| 22:00 | 10 Units | 22:00 | 9 Unit |

### Example III is Shown in Tables 10A-10B Below

A single daily basal injection is received and transformed into two daily basal injections.

| Current basal plan | | Recommended basal plan | |
|---|---|---|---|
| Time | Amount | Time | Amount |
| 08:00 | 20 Units | 08:00 | 15 Units |
| | | 22:00 | 7 Unit |

### Example IV is Shown in Tables 11A-11B Below

Two daily basal injection plans are received and transformed into a single injection plan.

| Current basal plan | | Recommended basal plan | |
|---|---|---|---|
| Time | Amount | Time | Amount |
| 08:00 | 20 Units | 08:00 | 21 Units |
| 22:00 | 10 Units | | |

### Bolus Plan

There are three different kinds of bolus plans:

1. Carb counting is shown in Tables 12A-12B below:

TABLE 12A
Current bolus plan

| | Morning | Afternoon | Evening | Night |
|---|---|---|---|---|
| | (05:00- | (11:00- | (17:00- | (22:00- |
| | 11:00) | 17:00) | 22:00) | 05:00) |
| Carbs ratio | 6 | 5 | 5 | 7.5 |
| (g/U) | | | | |
| Correction | 30 | 30 | 30 | 30 |
| factor | | | | |
| (mg/dL/U) | | | | |
| Blood | 160 | 160 | 160 | 160 |
| Glucose | | | | |
| target | | | | |
| (mg/dL) | | | | |

TABLE 12B
Recommended bolus plan

| | Morning | Afternoon | Evening | Night |
|---|---|---|---|---|
| | (05:00- | (11:00- | (17:00- | (22:00- |
| | 11:00) | 17:00) | 22:00) | 05:00) |
| Carbs ratio (g/U) | 6 | 4.5 | 4.5 | 7.5 |
| Correction | 25 | 30 | 30 | 30 |
| factor (mg/dL/U) | | | | |
| Blood Glucose | 150 | 150 | 150 | 150 |
| target (mg/dL) | | | | |

The times of the plan day periods may be adjusted and, in some cases, merged.

2. Sliding scale (fixed dose+correction) is shown in Tables 13A-13H below:

TABLE 13A
Current bolus plan
Morning (5AM-11AM)

| From | To | Amount |
|---|---|---|
| (mg/dL) | (mg/dL) | (Units) |
| 0 | 100 | 4 |
| 101 | 150 | 5 |
| 151 | 200 | 6 |
| 200 | 250 | 7 |
| 300 | High | 8 |

TABLE 13B
Recommended bolus plan

| From | To | Amount |
|---|---|---|
| (mg/dL) | (mg/dL) | (Units) |
| 0 | 100 | 5 |
| 101 | 150 | 6 |
| 151 | 200 | 7 |
| 200 | 250 | 8 |
| 300 | High | 9 |

TABLE 13C
Afternoon (11AM-5PM)

| From | To | Amount |
|---|---|---|
| (mg/dL) | (mg/dL) | (Units) |
| 0 | 100 | 6 |
| 101 | 150 | 7 |
| 151 | 200 | 8 |
| 200 | 250 | 9 |
| 300 | High | 10 |

TABLE 13D

| From | To | Amount |
|---|---|---|
| (mg/dL) | (mg/dL) | (Units) |
| 0 | 100 | 6 |
| 101 | 150 | 7 |
| 151 | 200 | 8 |
| 200 | 250 | 9 |
| 300 | High | 10 |

TABLE 13E
Evening (5PM-10PM)

| From | To | Amount |
|---|---|---|
| (mg/dL) | (mg/dL) | (Units) |
| 0 | 100 | 5 |
| 101 | 150 | 6 |
| 151 | 200 | 7 |
| 200 | 250 | 8 |
| 300 | High | 9 |

TABLE 13F

| From | To | Amount |
|---|---|---|
| (mg/dL) | (mg/dL) | (Units) |
| 0 | 100 | 6 |
| 101 | 150 | 7 |
| 151 | 200 | 8 |
| 200 | 250 | 9 |
| 300 | High | 10 |

TABLE 13G
Night (10PM-5AM)

| From | To | Amount |
|---|---|---|
| (mg/dL) | (mg/dL) | (Units) |
| 0 | 100 | 5 |
| 101 | 150 | 6 |
| 151 | 200 | 7 |
| 200 | 250 | 8 |
| 300 | High | 9 |

TABLE 13H

| From | To | Amount |
|---|---|---|
| (mg/dL) | (mg/dL) | (Units) |
| 0 | 100 | 6 |
| 101 | 150 | 7 |
| 151 | 200 | 8 |
| 200 | 250 | 9 |
| 300 | High | 10 |

3. Meal estimation+correction factor is shown in Tables 14A-14B below:

TABLE 14A
Current bolus plan

| | Morning | | | Afternoon | | |
|---|---|---|---|---|---|---|
| | (05:00-11:00) | | | (11:00-17:00) | | |
| | Low | Medium | High | Low | Medium | High |
| Meal | carb | carb | carb | carb | carb | carb |
| Estimation (U) | 5 | 6 | 7 | 5 | 6 | 7 |
| Correction | 30 | | | 30 | | |
| Factor | | | | | | |
| (md/dL/U) | | | | | | |
| Blood Glucose | 120 | | | 120 | | |
| Target (mg/dL) | | | | | | |
| | Evening | | | Night | | |
| | (17:00-22:00) | | | (22:00-05:00) | | |
| | Low | Medium | High | Low | Medium | High |
| Meal | carb | carb | carb | carb | carb | carb |
| Estimation (U) | 4 | 5 | 6 | 3 | 4 | 5 |
| Correction | 50 | | | 50 | | |
| Factor | | | | | | |
| (md/dL/U) | | | | | | |
| Blood Glucose | 120 | | | 120 | | |
| Target (mg/dL) | | | | | | |

TABLE 14B
Recommended bolus plan

| | Morning | | | Afternoon | | |
|---|---|---|---|---|---|---|
| | (05:00-11:00) | | | (11:00-17:00) | | |
| | Low | Medium | High | Low | Medium | High |
| | carb | carb | carb | carb | carb | carb |
| Meal | 6 | 7 | 8 | 5 | 6 | 7 |
| Estimation (U) | | | | | | |
| Correction | 30 | | | 30 | | |
| Factor | | | | | | |
| (md/dL/U) | | | | | | |
| Blood Glucose | 120 | | | 120 | | |
| Target (mg/dL) | | | | | | |
| | Evening | | | Night | | |
| | (17:00-22:00) | | | (22:00-05:00) | | |
| | Low | Medium | High | Low | Medium | High |
| | carb | carb | carb | carb | carb | carb |
| Meal | 5 | 6 | 7 | 3 | 4 | 5 |
| Estimation (U) | | | | | | |

| | | |
|---|---|---|
| Correction | 40 | 40 |
| Factor | | |
| (md/dL/U) | | |
| Blood Glucose | 120 | 120 |
| Target (mg/dL) | | |

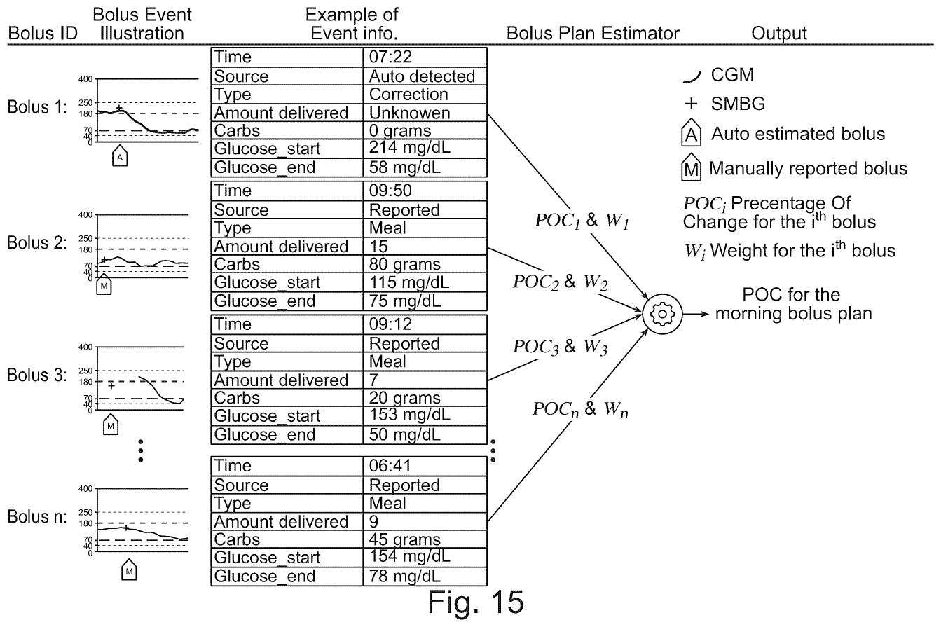

Reference is made now to FIG. 15 showing an example of a possible internal analysis performed based on bolus events found in the morning period. In some embodiments, the control unit may be configured to collect a plurality of bolus events (e.g. from I to n) and generate a weight and percentage of change (POC) for each of them. The control unit may also comprise a bolus plan estimator being configured to calculate the POC. The final POC can apply to all types of bolus plans: for example, single POC in case of SSI plan and two separated and targeted POC for the CR and the CF, in case of a CR/CF plan.

In some embodiments, some bolus events may be filtered out from the bolus plan estimation. In this specific example of FIG. 15, at the morning hours, the weight given to bolus #2 can be zero and therefore it can be filtered out from the POC estimation. The reason for the zero weight may be the discrepancy found between the amount delivered (15 units) and the expected amount based on the inputs (80 grams of carbohydrates and glucose level of 115 mg/dL).

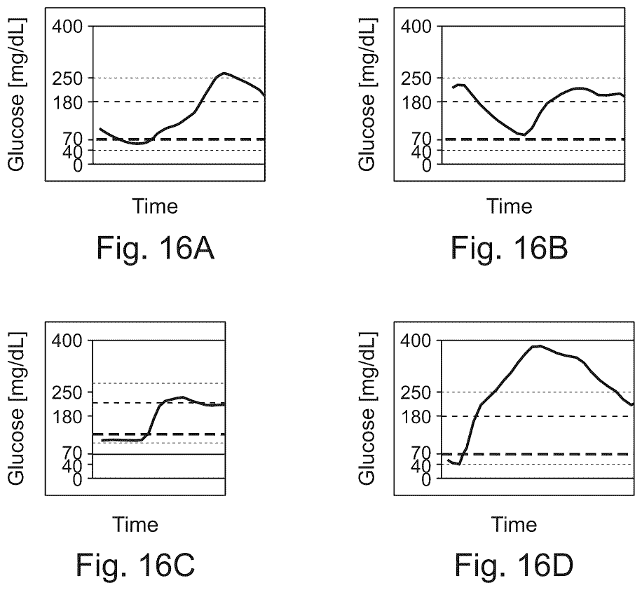

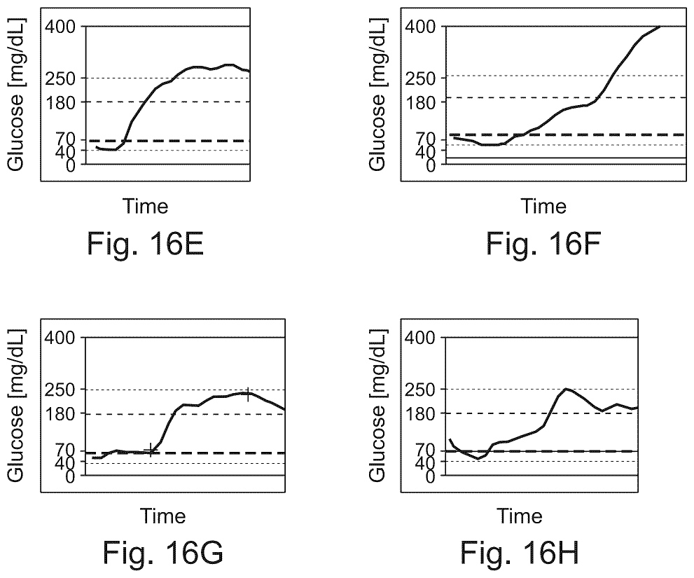

In some embodiments, the monitoring system 100 of FIG. 1 is able to perform behavioral pattern analysis. This technique may enable to provide to the patient an adapted behavioral tip. Reference is made now to FIGS. 16A-16H illustrating an example in which the system detects a behavioral pattern of overtreating hypoglycemia. In this example, the system analyzed the behavioral patterns and identified a hyperglycemia shortly after a hypoglycemia event. This identification can trigger a behavioral tip for the patient to avoid overtreating hypoglycemia events.

In some embodiments, the finding of a behavioral pattern such as exampled above, may prevent a proposed dose change based on the basal and/or bolus estimation changes. For example, finding of repeated overtreating hypoglycemia in the morning can prevent the system from outputting an increase in the insulin dose for boluses at that time.

1. A monitoring system for use with diabetic treatment management based on insulin injections, the monitoring system comprising:
a communication interface configured and operable to permit access to retrospective raw log glucose data being indicative of blood glucose levels over a certain time period; wherein the certain time period being at least one daily period; and at least one of an existing patient-specific insulin injections treatment plan or user data being indicative of at least one of event or insulin delivery over the certain time period, wherein the event comprises at least one of sleep, meal, exercise, disease event, or rest; wherein said communication interface is configured to permit input of at least a part of the retrospective raw log glucose data directly from an external device; wherein the external device comprises at least one of the following: a measurement device, a storage device, and an injection device; and
a control unit configured and operable for receiving and processing the retrospective raw glucose log data with at least one of the existing insulin injections treatment plan or the user data and automatically determining a recommended patient-specific insulin injections treatment plan based only on basal data or a recommended patient-specific Multiple Daily Injections (MDI) treatment plan, wherein the recommended patient-specific insulin injections treatment plan comprises individualized insulin dosing injection parameters data over the certain time period, wherein the individualized insulin dosing injection parameters data comprises at least one of a basal plan, a bolus plan or suggestions for personalized diabetes management tips.

2. The monitoring system of claim 1, wherein the control unit is configured and operable for automatically determining at least one of (i) the basal plan including an amount and timing of long-acting insulin dosage, or (ii) bolus plan including an amount of short acting insulin dosage, wherein the control unit is configured and operable for automatically determining an amount of long acting insulin dose comprising analyzing the blood glucose pattern to define a certain time of injecting the long-acting insulin dose and a certain number of required doses of the basal insulin.

3. (canceled)

4. The monitoring system of claim 1, wherein the control unit is configured and operable for automatically determining the bolus plan including (1) carbohydrate ratio (CR) according to the time of day, correction factor (CF) and glucose target, and/or (2) fixed dose, CF and glucose target, and/or (3) meal size estimation, CF and glucose target and/or (4) Sliding Scale (SSI) over the certain period of time.

5. The monitoring system of claim 1, wherein the control unit is configured and operable for automatically determining and providing the suggestions for personalized diabetes management tips to at least one of a patient or a physician, wherein the suggestions for the personalized diabetes management tips comprises textual output data being indicative of at least one of timing of meal boluses, bolus delivery compliance, or a treatment alert.

6. The monitoring system of claim 1, wherein said communication interface comprises a user interface to permit user input of the user data comprising data being indicative of insulin delivery over a certain time, meals consumed, at least a part of the raw log glucose data, exercise intensity, or any other textual data being indicative of the user's condition.

7. (canceled)

8. (canceled)

9. (canceled)

10. The monitoring system of claim 1, wherein said control unit is configured and operable to correlate between the raw log glucose data, insulin records and the existing patient-specific insulin injections treatment plan to validate the existing patient-specific insulin injections treatment plan.

11. The monitoring system of claim 1, wherein said control unit is configured and operable to receive and process the measured data to identify the user data being indicative of insulin delivery over the certain time period or meals consumed during the certain time period.

12. The monitoring system of claim 1, wherein said control unit is configured and operable to process the raw log glucose data to identify a start point and an end point of a certain inactive time period being indicative of an absence of insulin delivery and meal consumption.

13. The monitoring system of claim 1, wherein said control unit is configured and operable to process the raw log glucose data to thereby identify, in the raw log glucose data, event data being indicative of at least one of insulin delivery or at least one meal consumed in a certain time period; classifying the event data in different events, either relating or not relating to the existing patient-specific insulin injections treatment plan, and filtering out the events not relating to the existing patient-specific insulin injections treatment plan.

14. The monitoring system of claim 13, wherein said control unit is configured and operable to automatically determine a patient-specific bolus treatment plan based on event data by giving different weights to the classified events.

15. A method of automatic monitoring of diabetes-related treatment of a patient based on insulin injections, the method comprising:
receiving retrospective raw log glucose data being indicative of at least one blood glucose pattern defining blood glucose levels over a certain time period; wherein the certain time period being at least one daily period; and at least one of an existing patient-specific insulin injections treatment plan or a user data comprising at least one of data being indicative of insulin delivery over the certain time period, or of data being indicative of meals consumed during the certain time period; wherein receiving measured data comprises receiving input of at least a part of the retrospective raw log glucose data from an external device; and
processing the retrospective raw log glucose data with at least one of the existing insulin injections treatment plan or the user data, to automatically determine a recommended patient-specific insulin injections treatment plan based only on basal data or a recommended patient-specific Multiple Daily Injections (MDI) treatment plan; wherein the recommended patient-specific insulin injections treatment plan comprises individualized insulin dosing injection parameters data over the certain time period, wherein the individualized insulin dosing injection parameters data comprises at least one of a basal plan a bolus plan or suggestions for personalized diabetes management tips.

16. The method of claim 15, wherein automatically determining a patient-specific insulin injections treatment plan comprises automatically determining a basal plan including at least one of an amount of long-acting insulin dosage, timing of insulin delivery or number of required doses of basal insulin:
wherein automatically determining an amount of long-acting insulin dosage comprises analyzing the glucose pattern to define a certain time of injecting the long-acting insulin dose and a certain number of required dose of the basal insulin, and
wherein automatically determining the bolus plan includes determining (1) CR CF and glucose target according to the time of day and/or (2) fixed dose, CF and glucose target, and/or (3) meal size estimation, CF and glucose target and/or (4) SSI over the certain period of time.

17. (canceled)

18. (canceled)

19. The method of claim 15, wherein automatically determining the personalized diabetes management tips comprises providing the personalized diabetes management tips to at least one of the patient or a physician, wherein the suggestions for personalized diabetes management tips comprises textual output data comprising at least one of timing of meal boluses, bolus delivery compliance, overtreating hypoglycemia, bolus stacking, or other treatment alert.

20. The method of claim 15, wherein receiving user data comprises receiving user input of the user data comprising data being indicative of at least of the insulin delivery over the certain time, meals consumed, at least a part of the raw log glucose data, exercise intensity, or any other textual data being indicative of the user's condition.

21. The method of claim 15, wherein receiving measured data comprises receiving measured data from a plurality of external devices, wherein processing the raw log glucose data comprises synchronizing between the measured data generated by the plurality of external devices.

22. (canceled)

23. The method of claim 15, wherein processing the raw log glucose data and the user data comprises synchronizing between the raw log glucose data and the user data,
wherein the method further comprises correlating between the raw log glucose data, insulin records and the existing patient-specific insulin injections treatment plan to validate the existing patient-specific insulin injections treatment plan.

24. (canceled)

25. The method of claim 15, wherein said processing the raw log glucose data comprises receiving and processing the measured data to identify the user data being indicative of insulin delivery over the certain time period or meals consumed during the certain time period.

26. The method of claim 15, wherein said processing the raw log glucose data comprises identifying a start point and an end point of a certain inactive time period being indicative of an absence of insulin delivery and meal consumption.

27. The method of claim 15, wherein said processing the raw log glucose data comprises identifying, in the raw log glucose data, event data being indicative of at least one of insulin delivery or at least one meal consumed in a certain time period; classifying the event data in different events relating or not to the existing patient-specific insulin injections treatment plan, and filtering out the events not relating to the existing patient-specific insulin injections treatment plan: wherein said automatically determining of a patient-specific insulin injections treatment plan comprises processing the event data by giving different weights to the classified event data.

28. (canceled)

29. A computer system comprising a communication interface configured and operable to permit access to retrospective raw log glucose data being indicative of blood glucose levels over a certain time period via at least one communication network to a server, and a storage medium on which a computer program is recordable in a machine-readable format and being configured and operable, when being accessed, to carry out the method according to claim 15, the computer program being downloadable to the storage medium or in electronic form from a server over a network.
